SEQUENCE LISTING (35 sequences)

SEQ ID NO: 1 - protein, 120 aa, Artificial Sequence
  synthesized
QVQLVQSGAEVKKPGASVKVSCKVSAYAFSSSWMNWVRQAPGKGLEWMGRIYPRDGDTNYNGKFKGRVTMTADTSTDTAYMELSSLRSEDTAVYYCAREGDGYYWYFDVWGQGTTVTVSS

SEQ ID NO: 2 - protein, 107 aa, Artificial Sequence
  synthesized
EIVLTQSPATLSLSPGERATLSCRASQSIRNYLHWYQQKPGEAPRLLIYYASQSISGIPARFSGSGSGTDFTLTISSLETEDFAMYYCQHSNSWPLTFGQGTKLEIK

SEQ ID NO: 3 - protein, 116 aa, Artificial Sequence
  synthesized
EVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSA

SEQ ID NO: 4 - protein, 107 aa, Artificial Sequence
  synthesized
DIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIK

SEQ ID NO: 5 - protein, 116 aa, Artificial Sequence
  synthesized
EVQLLESGGGLVQPGGSLRLSCAASGFTFSSYAMSWVRQAPGKGLEWVSVIDSNGGSTYYPDTVKDRFTISRDNSKNTLYLQMNSLRAEDTAVYYCAKYTNLGAYWGQGTLVTVSS

SEQ ID NO: 6 - protein, 107 aa, Artificial Sequence
  synthesized
DIQMTQSPSSLSASVGDRVTITCRASQDISGYLNWYQQKPGKAPKLLIYTTSTLDSGVPSRFSGSGSGTDFTLTISSLQPEDFATYYCLQYASSPFTFGGGTKVEIK

SEQ ID NO: 7 - protein, 120 aa, Artificial Sequence
  synthesized
QVQLVQSGAEVKKPGASVKVSCKVSGYTFTHYWMHWVRQRPGKGLEWMGEIDPFDSYTYYNQKFKGRVTMTVDTSSDTAYMELSSLRSEDTAVYYCARPLPGTGWYFDVWGQGTTVTVSS

SEQ ID NO: 8 - protein, 108 aa, Artificial Sequence
  synthesized
EIVLTQSPTTLSLSPGERATLSCSASSSISSTYLHWYQQKPGFPPRLLIYGTSNLASGIPACFSGSGSGTDFTLTISSLEAEDFAVYYCQQGSSLPFTFGQGTKLEIK

SEQ ID NO: 9 - protein, 120 aa, Artificial Sequence
  synthesized
EVQLVESGGGLVQPGGSLRLSCAASGFTFSTYTMSWVRQTPAKGLVWVSTINSDGYNIYYSDSMKGRFTISRDNAKYTLYLQMNSLRAEDTAMYYCARCSYYSYDYFDYWGQGTLVTVSS

SEQ ID NO: 10 - protein, 107 aa, Artificial Sequence
  synthesized
DIQMTQSPSSLSASVGDRVTITCRASENIDNYLAWYQQKQGKVPKLLIYAATNLADGMPSRFSGSGSGTDFTLTISSLQPEDVATYYCQHYYSNQLTFGQGTKLEIK

SEQ ID NO: 11 - protein, 122 aa, Artificial Sequence
  synthesized
QVQLVQSGAEVKKPGASVKVSCKASGYTFTDFYMNWVRQAPGQGLEWMGRVNPSNGDTNYNQKFKGRVTSTRDTSISTAYMELSRLRSDDTAVYYCARERIYYGISWYFDVWDTGTTVTVSS

SEQ ID NO: 12 - protein, 108 aa, Artificial Sequence
  synthesized
DIQMTQHSPSSLSASVGDRVTITCRASGNIHNYLAWYQQKPGKAPKLLLYNAKTLAEGVPSRFSGSGSGTDYTLTISSLQPEDFATYYCHHYYSTPPTFGQGTKLEIK

SEQ ID NO: 13 - protein, 598 aa, Artificial Sequence
  synthesized
MEFGLSWVFLVALLRGVQCEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKPAPAPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 594 aa, Artificial Sequence
  synthesized
MEFGLSWVFLVALLRGVQCEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKPAPAPASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 15 - protein, 598 aa, Artificial Sequence
  synthesized
MEFGLSWVFLVALLRGVQCEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKPAPAPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 376 aa, Artificial Sequence
  synthesized
MEFGLSWVFLVALLRGVQCQVQLVQSGAEVKKPGASVKVSCKVSAYAFSSSWMNWVRQAPGKGLEWMGRIYPRDGDTNYNGKFKGRVTMTADTSTDTAYMELSSLRSEDTAVYYCAREGDGYYWYFDVWGQGTTVTVSSGGGGSGGGGSGGGGSEIVLTQSPATLSLSPGERATLSCRASQSIRNYLHWYQQKPGEAPRLLIYYASQSISGIPARFSGSGSGTDFTLTISSLETEDFAMYYCQHSNSWPLTFGQGTKLEIKPAGGGGSGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 372 aa, Artificial Sequence
  synthesized
MEFGLSWVFLVALLRGVQCEVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSAGGGGSGGGGSGGGGSDIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIKPAGGGGSGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 716 aa, Artificial Sequence
  synthesized
MEFGLSWVFLVALLRGVQCEVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPAGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 19 - protein, 712 aa, Artificial Sequence
  synthesized
MEFGLSWVFLVALLRGVQCEVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKPAGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 20 - protein, 716 aa, Artificial Sequence
  synthesized
MEFGLSWVFLVALLRGVQCEVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPAGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 21 - protein, 234 aa, Artificial Sequence
  synthesized
MRLPAQLLGLLMLWVSGSSGDIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 497 aa, Artificial Sequence
  synthesized
MEFGLSWVFLVALLRGVQCEVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSAGGGGSGGGGSGGGGSDIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIKPAGGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 493 aa, Artificial Sequence
  synthesized
MEFGLSWVFLVALLRGVQCEVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSAGGGGSGGGGSGGGGSDIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIKPAGGGGGSERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 24 - protein, 497 aa, Artificial Sequence
  synthesized
MEFGLSWVFLVALLRGVQCEVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSAGGGGSGGGGSGGGGSDIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIKPAGGGGGSEPKSCDKTHTCPPCPAPELLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 242 aa, Artificial Sequence
  synthesized
QVQLVQSGAEVKKPGASVKVSCKVSAYAFSSSWMNWVRQAPGKGLEWMGRIYPRDGDTNYNGKFKGRVTMTADTSTDTAYMELSSLRSEDTAVYYCAREGDGYYWYFDVWGQGTTVTVSSGGGGSGGGGSGGGGSEIVLTQSPATLSLSPGERATLSCRASQSIRNYLHWYQQKPGEAPRLLIYYASQSISGIPARFSGSGSGTDFTLTISSLETEDFAMYYCQHSNSWPLTFGQGTKLEIK

SEQ ID NO: 26 - protein, 238 aa, Artificial Sequence
  synthesized
EVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSAGGGGSGGGGSGGGGSDIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIK

SEQ ID NO: 27 - protein, 238 aa, Artificial Sequence
  synthesized
EVQLLESGGGLVQPGGSLRLSCAASGFTFSSYAMSWVRQAPGKGLEWVSVIDSNGGSTYYPDTVKDRFTISRDNSKNTLYLQMNSLRAEDTAVYYCAKYTNLGAYWGQGTLVTVSSGGGGSGGGGSGGGGSDIQMTQSPSSLSASVGDRVTITCRASQDISGYLNWYQQKPGKAPKLLIYTTSTLDSGVPSRFSGSGSGTDFTLTISSLQPEDFATYYCLQYASSPFTFGGGTKVEIK

SEQ ID NO: 28 - protein, 243 aa, Artificial Sequence
  synthesized
QVQLVQSGAEVKKPGASVKVSCKVSGYTFTHYWMHWVRQRPGKGLEWMGEIDPFDSYTYYNQKFKGRVTMTVDTSSDTAYMELSSLRSEDTAVYYCARPLPGTGWYFDVWGQGTTVTVSSGGGGSGGGGSGGGGSEIVLTQSPTTLSLSPGERATLSCSASSSISSTYLHWYQQKPGFPPRLLIYGTSNLASGIPACFSGSGSGTDFTLTISSLEAEDFAVYYCQQGSSLPFTFGQGTKLEIK

SEQ ID NO: 29 - protein, 242 aa, Artificial Sequence
  synthesized
EVQLVESGGGLVQPGGSLRLSCAASGFTFSTYTMSWVRQTPAKGLVWVSTINSDGYNIYYSDSMKGRFTISRDNAKYTLYLQMNSLRAEDTAMYYCARCSYYSYDYFDYWGQGTLVTVSSGGGGSGGGGSGGGGSDIQMTQSPSSLSASVGDRVTITCRASENIDNYLAWYQQKQGKVPKLLIYAATNLADGMPSRFSGSGSGTDFTLTISSLQPEDVATYYCQHYYSNQLTFGQGTKLEIK

SEQ ID NO: 30 - protein, 244 aa, Artificial Sequence
  synthesized
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 31 - protein, 469 aa, Artificial Sequence
  synthesized
QVQLVQSGAEVKKPGASVKVSCKVSAYAFSSSWMNWVRQAPGKGLEWMGRIYPRDGDTNYNGKFKGRVTMTADTSTDTAYMELSSLRSEDTAVYYCAREGDGYYWYFDVWGQGTTVTVSSGGGGSGGGGSGGGGSEIVLTQSPATLSLSPGERATLSCRASQSIRNYLHWYQQKPGEAPRLLIYYASQSISGIPARFSGSGSGTDFTLTISSLETEDFAMYYCQHSNSWPLTFGQGTKLEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 32 - protein, 427 aa, Artificial Sequence
  synthesized
EVQLVESGGGLVQPGGSLRLSCAASGFTFSTYTMSWVRQTPAKGLVWVSTINSDGYNIYYSDSMKGRFTISRDNAKYTLYLQMNSLRAEDTAMYYCARCSYYSYDYFDYWGQGTLVTVSSGGGGSGGGGSGGGGSDIQMTQSPSSLSASVGDRVTITCRASENIDNYLAWYQQKQGKVPKLLIYAATNLADGMPSRFSGSGSGTDFTLTISSLQPEDVATYYCQHYYSNQLTFGQGTKLEIKGAPTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 33 - protein, 352 aa, Artificial Sequence
  synthesized
EVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSAGGGGSGGGGSGGGGSDIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCEL

SEQ ID NO: 34 - protein, 756 aa, Artificial Sequence
  synthesized
MEFGLSWVFLVALLRGVQCQVQLVESGGGVVQPGRSLRLSCAASGFTFSDYYMYWVRQAPGKGLEWVASISFDGTYTYYTDRVKGRFTISRDNSKNTLYLQMNSLRAEDTAVYYCARDRPAWFPYWGQGTLVTVSAGGGGSGGGGSGGGGSGDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPLTFGAGTKLELKPAGGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGAPGGGSGEPKSSDKTHTCPPCPAPELLGGPSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 35 - protein, 756 aa, Artificial Sequence
  synthesized
MEFGLSWVFLVALLRGVQCQVQLVESGGGVVQPGRSLRLSCAASGFTFSDYYMYWVRQAPGKGLEWVASISFDGTYTYYTDRVKGRFTISRDNSKNTLYLQMNSLRAEDTAVYYCARDRPAWFPYWGQGTLVTVSAGGGGSGGGGSGGGGSGDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPLTFGAGTKLELKPAGGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGAPGGGSGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2021/0115152 A1
(43) Pub. Date: Apr. 22, 2021

(54) COMPOSITION OF BISPECIFIC ANTIBODIES AND METHOD OF USE THEREOF

(71) Applicant: ARBELE LIMITED, Hong Kong (CN)

(72) Inventors: Donald E. STAUTON, Kirkland, WA (US); John Moonching LUK, BELLEVUE, WA (US)

(21) Appl. No.: 17/055,561

(22) PCT Filed: May 15, 2019
(86) PCT No.: PCT/US2019/032528

§ 371 Date: Nov. 13, 2020

Related U.S. Application Data
(60) Provisional application No. 62/672,325, filed on May 16, 2018.

(51) Int. Cl. C07K 16/30; C07K 16/28; A61P 35/00; A61K 47/68; A61K 33/243; A61K 31/7068; A61K 31/4745; A61K 39/395

(52) U.S. Cl. CPC C07K 16/30; A61K 2039/505; A61P 35/00; A61K 47/6851; A61K 47/6803; A61K 33/243; A61K 31/7068; A61K 31/4745; A61K 39/39541; A61K 39/39558; C07K 2317/31; C07K 2317/622; C07K 2317/522; C07K 2317/53; C07K 2317/524; C07K 2317/526; C07K 2317/24; C07K 2317/73; C07K 2317/74; C07K 16/2809

(57) ABSTRACT

Embodiments of the present disclosure relate to the composition of bispecific antibodies against cadherin-17 and CD3 and method of using the antibodies for cancer treatment.

### CROSS REFERENCE TO RELATED APPLICATIONS

This application claims the benefit of the filing date of U.S. Provisional Application Ser. No. 62/672,325 filed May 16, 2018 under 35 U.S.C. 119(e), the entire disclosures of which are incorporated by reference herein.

### TECHNICAL FIELD

The present disclosure generally relates to the technical field of cancer immunotherapy, and more particularly to cadherin-17 (CDH17) specific antibodies and cytotoxic cells for cancer treatment.

### BACKGROUND

Unless otherwise indicated herein, the materials described in this section are not prior art to the claims in this application and are not admitted to be prior art by inclusion in this section.

Despite the recent advances in drug discovery and clinical imaging, cancer remains one of the deadliest diseases in humans. Our understandings on how tumor initiates, survives under stress, colonizes/metastasizes to distant organs and sites, and becomes resistant to drugs are still limited. The American Cancer Society estimated new cases of cancer in the US in 2014 is 1.6 million, with no approved curative treatment for most of the predominant types of cancer.

Gastrointestinal (GI) cancers (colorectal, gastric, pancreatic, esophageal, bile duct and liver) are leading causes of morbidity and mortality worldwide. Colorectal carcinoma (CRC) alone represents approximately 10% of all cancer diagnosis and is the second leading cause of cancer deaths world-wide. In China, liver and stomach cancers are among the most lethal of malignancies worldwide and over half of the incidences diagnosed, causing>1.42 million deaths per year globally, which are believed attributable to the viral/bacterial endemic (Hepatitis B virus [HBV] and Helicobacter pylori infections), chemical intoxications, environmental pollutions and food contaminations. There are no effective therapies. New biomarkers and therapeutic targets are thus needed for potential drug development against these aggressive cancers. A proven molecular targeting agent that can eliminate or repress the growth of these cancers will have important clinical value and significant market impact. These tumors can be resected effectively by surgery if the diseases are diagnosed in early stages. Unfortunately, and very often, most of GI cancers are asymptomatic and detected at very advanced stages when presented in the clinic. Without effective treatment, these patients die shortly after the diagnosis or relapse after salvage therapies.

CDH17 is a prominent cancer biomarker characterized by its overexpression in both liver and stomach cancers but not normal tissues from healthy adults. Anti-CDH17 monoclonal antibody displays the growth inhibitory effect on liver and stomach tumour cells. CDH17 is highly expressed in metastatic cancers, and the blockage of CDH17 expression and functions can markedly reduce lung metastasis of hepatocellular carcinoma (HCC). These observations indicate that humanized anti-CDH17 antibody may be developed as antibody therapeutics for treating cancer patients with indication of CDH17 biomarker in tumour tissues and/or in serum samples.

In contrast to antibody therapeutics characterized by the binding of monoclonal antibody to cancer cells, multi-specific antibody therapeutics may bind to T cells and mediate the cytotoxicity towards cancer cells. Bispecific antibodies are effective for treating hematologic malignancies but show limited success targeting solid tumours. The possible obstacles may be that activated cytotoxic immune cells lack suitable biomarkers and the solid tumour cells are less accessible.

### SUMMARY

The disclosure provides compositions of multi-specific antibodies and cytotoxic cells targeting CDH17, and methods for treating cancers with the compositions and antibodies (or fragment thereof) disclosed herein.

In one aspect, the disclosure relates to the composition of multi-specific antibodies targeting both a gastrointestinal specific biomarker and CD3. In some embodiments, the antibodies are CDH17xCD3 bispecific antibodies. These antibodies may activate T cells and safely target CDH17-positive cells. In one embodiment, CDH17xCD3 bispecific antibodies may be used clinically for treating patients with CDH17 positive cancers.

In one embodiment, the disclosure provides an antibody having specificity for CDH17, comprising a heavy or light chain amino acid sequence having a homology at least 70%, 80%, 90%, 95%, 96%, 97%, 98%, 99%, or 100% (or any other number in between) with an amino acid sequence selected from SEQ ID NO: 15-33.

In some embodiments, the antibodies are monoclonal antibodies. In one embodiment, the monoclonal antibody may be a mouse antibody, a humanized antibody, or a human antibody. In some embodiments, the monoclonal antibody may be a human antibody isolated from a phage library screen.

In some embodiments, the antibody may include a variable region of light chain (VL), a variable region of heavy chain (VH), or a combination thereof. In one embodiment, the VL may include an amino acid sequence having a homology of at least 70%, 80%, 85%, 90% m 95%, 98%, 99% or 100% (or any other number in between) with an amino acid sequence selected from SEQ ID NO: 2, 4, 6, 8, 10 and 12. In some embodiments, the VH may include an amino acid sequence having a homology of at least 70%, 80%, 85%, 90% m 95%, 98%, 99% or 100% (or any other number in between) with an amino acid sequence selected from SEQ ID NO: 1, 3, 5, 7, 9 and 11.

In some embodiments, the antibody may include a conjugated cytotoxic moiety. In some embodiments, the conjugated cytotoxic moiety may include irinotecan, auristatins, PBDs, maytansines, amantins, spliceosome inhibitors, ora combination thereof. In some embodiments, the conjugated cytotoxic moiety may include a chemotherapeutic agent.

In some embodiments, the antibody is a bispecific antibody.

In some embodiments, the antibody may include specificity for a cell receptor from a cytotoxic T or NK cell. In some embodiments, the antibody is a bispecific antibody having specificity for both CDH17 and CD3. In some embodiments, the cell receptor may include KIR2D52, KIR2D53, KIR2D54, KIR2D55, KIR3D51, CD16a, CD27, CD94, CD96, CD100, CD160, CD244, NKp30, NKp44, NKp46, NKp80, NKG2D, DNAM1, CRTAM, PSGL1, CEACAM1, NTB-A, SLAMF7, OX40, CD137, ICOS, CD28, TIM1, and TIM3, or a derivative or combination thereof.

In some embodiments, the antibody may include a first single-chain variable fragment (scFv) having specificity for CDH17 and a second single-chain variable fragment (scFv) having specificity for CD3 or TROP2. In one embodiment, the first scFv may include a first VH (variable heavy chain) and a first VL (variable light chain). In one embodiment, the second scFv may include a second VH and a second VL. In some embodiments, the first VH may include an amino acid sequence having a homology of at least 70%, 80%, 85%, 90%, 95%, 98%, 99% or 100% (or any other number in between) with an amino acid sequence selected from SEQ ID NO:1, 3, 5, 7. In some embodiments, the first VL may include an amino acid sequence having a homology of at least 70%, 80%, 85%, 90%, 95%, 98%, 99% or 100% (or any other number in between) with an amino acid sequence selected from SEQ ID NO:2, 4, 6, 8.

In some embodiments, the second VH may include a corresponding portion of an amino acid sequence having a homology at least 70%, 80%, 85%, 90%, 95%, 98%, 99% or 100% (or any other number in between) with the amino acid SEQ ID NO: 9, 11, 13.

In some embodiments, the second VL may include a corresponding portion of an amino acid sequence having a homology of at least 70%, 80%, 85%, 90%, 95%, 98%, 99% or 100% (or any other number in between) with the amino acid SEQ ID NO: 10, 12, 14.

In some embodiments, the antibody may have a specificity for an immune checkpoint inhibitor. In some embodiments, the checkpoint inhibitor may include PD-1, PD-L1, CTL-A4, TIM3, LAG3, BTLA, CD96, TIGIT, CD226 or VISTA, or a combination thereof.

In some embodiments, the antibody may have a specificity for an angiogenic factor. In some embodiments, the angiogenic factor may include VEGF.

In some embodiments, the antibody may be configured to antagonize the binding of the RGD site in CDH17 domain 6 to integrin. In some embodiments, the integrin may include alpha2beta1.

In some embodiments, the antibody may be configured to bind CDH17 ectodomain domain 5, domain 6 or domain 7 to antagonize CDH17 shedding.

In some embodiments, the antibody is a monoclonal antibody.

Some embodiments relate to an IgG heavy chain for an antibody. In one embodiment, the antibody may have an IgG having chain having a homology at least 70%, 80%, 85%, 90%, 95%, 98%, 99% or 100% (or any other number in between) with SEQ ID NO: 15, 16, 17, 20, 21, 22, 24, 25. 26, 28, 29, 30, 31, 32, 33.

Some embodiments relate to a light chain for an antibody. In one embodiment, the antibody may have a light chain having an amino acid sequence having a homology of at least 70%, 80%, 85%, 90%, 95%, 98%, 99% or 100% (or any other number in between) with SEQ ID NO:18, 19, 23, 27.

Some embodiments relate to a variable domain for an antibody. In one embodiment, the variable domain may have an amino acid having a homology of at least 70%, 80%, 85%, 90%, 95%, 98%, 99% or 100% (or any other number in between) of with SEQ ID NO:1-14.

Some embodiments relate to a scFv or Fab having specificity for CDH17. In some embodiment, the antibody includes an amino acid sequence having a homology of at least 70%, 80%, 85%, 90%, 95%, 98%, 99% or 100% (or any other number in between) with an amino acid sequence selected from SEQ ID NO: 34, 35, 36, 37, 38, 39, 40.

In some embodiments, the scFv or Fab may include specificity for a cell receptor from a cytotoxic T or NK cell. In some embodiments, the scFv or Fab may include specificity for an immune checkpoint inhibitor. In some embodiments, the scFv or Fab may include specificity for an angiogenic factor.

Some embodiments relate to a T or NK cell having specificity for CDH17. In one embodiment, the T or NK cell may include a chimeric antigen receptor. In one embodiment, the chimeric antigen receptor may include an amino acid sequence having a homology of at least 70%, 80%, 85%, 90%, 95%, 98%, 99% or 100% (or any other number in between) with an amino acid sequence selected from SEQ ID NO: 41, 42, 43, 44, 45.

Some embodiments relate to an isolated nucleic acid encoding the antibody, the IgG heavy Chain, the light chain, the variable chain, or the scFv or Fab as described herein.

Some embodiments relate to an expression vector comprising the isolated nucleic acid disclosed herein. In some embodiments, the vector is expressible in a cell.

Some embodiments relate to a host cell comprising the nucleic acid as described herein. Some embodiments relate to a host cell comprising the expression vector as described herein. In some embodiments, the host cell is a prokaryotic cell or a eukaryotic cell.

In one aspect, the application provides pharmaceutical compositions for treating cancer. In one embodiment, the pharmaceutical composition includes an antibody and a cytotoxic agent.

In some embodiments, the cytotoxic agent may include cisplatin, gemcitabine, irinotecan, or an anti-tumor antibody.

In some embodiments, the pharmaceutical composition may include the antibody as described herein and a pharmaceutically acceptable carrier.

In a further aspect, the application provides methods for treating a subject having cancer. In one emboidment, the method incldues administering to the subject an effective amount of an antibody or T or NK cells. In some embodiments, the effective amount may be an amount that would treat cancer, alleviate symptom, change a biomarker to assist in treating the cancer, or a combination thereof. The subject may be human or an animal.

In some embodiments, the cancer may liver cancer, gastric cancer, colon cancer, pancreatic cancer, lung cancer, or a combination thereof.

The objectives and advantages of the disclosure may become apparent from the following detailed description of embodiments thereof in connection with the accompanying drawings.

### BRIEF DESCRIPTION OF THE DRAWINGS

Embodiments according to the present disclosure may now be described with reference to the FIGs, in which like reference numerals denote like elements.

FIG. 1 shows structural variants of example bispecific antibodies against CDH17 and CD3 designated as scFv₄-Ig or tB (tetraB), IgG-scFv or fL (full length), and taFv-Fc or Fc (Bite-Fc);

FIG. 2 shows the sequence alignment of example variable domains of humanized CDH17 antibodies in SEQ ID NO:1-8; TROP2 antibody in SEQ ID NO: 9 and 10; and CD3 antibody in SEQ ID NO: 11 and 12;

FIG. 3 shows the CDH17 expression in tumor cell lines of DLD-1 (colon cancer) and AGS (gastric cancer) using a CDH17xCD3 bispecific antibody as an example, ARB201 (h3G1Fc), and flow cytometry analysis;

FIG. 4 shows live cell image of ARB201 antibody directed retargeted T cell cytotoxicity to DLD-1 Spheroid; DLD-1 cells were stained with CellBrite™ Green and grown as spheroids; Cells were incubated for 48 hours in the presence or absence of PBMCs and/or ARB201 (Ab); Retargeted T cell cytotoxicity was monitored by red fluorescent staining of the dead target cells; Bright field, green: GFP filter set; red: PI filter set; and live cell images were acquired and analyzed with automated fluorescent imager;

FIG. 5 shows concentration response of ARB201 in 2D and 3D DLD-1 Models; DLD-1 cells were incubated with fresh PBMC in the presence of different concentrations of ARB201; DLD-1 cell death was evaluated at 48 hours; Retargeted T cell Cytotoxicity was monitored with dead red dye; IC₅₀ values were calculated using nonlinear regression fit data to a sigmoidal 4-point, 4-parameter log-logistic dose response model;

FIG. 6 shows concentration Response of ARB201 in 2D and 3D AGS Models; AGS cells were incubated with fresh PBMC in the presence of different concentrations of ARB201; AGS cell death was evaluated at 16 hrs; Retargeted T cell Cytotoxicity was monitored with dead red dye; IC₅₀ values were calculated using nonlinear regression fit data to a sigmoidal 4-point, 4-parameter log-logistic dose response model;

FIG. 7 shows schematic of ARB201 retargeted T cell cytotoxicity; A) ARB201 binds both T cells (red) and tumor cells (green) to support T cell contact with tumor target cells; B) bright field; and C) ARB201 binding to CD3/TCR stimulates a cytotoxic T cell response with the release of perforin and granzymes that create pores and trigger apoptosis, respectively;

FIG. 8 shows the binding of example CDH17xCD3 bispecific antibodies, h5G1fL and h5G4fL, to CDH17 as determined by ELISA;

FIG. 9 shows the binding of example CDH17xCD3 bispecific antibodies, h5G1fL and h5G4fL, to CD3 on Jurkat T cells;

FIG. 10 shows the binding of example CDH17xCD3 bispecific antibodies, h10G1fL and h10G4fL, to CDH17 as determined by ELISA;

FIG. 11 shows the binding of example CDh17xCD3 bispecific antibodies, h10G1fL and h10G4fL, to CD3 on Jurkat T cells;

FIG. 12 shows the binding of example CDH17xCD3 bispecific antibodies, h10G1tB and h10G4tB, to CDH17 as determined by ELISA;

FIG. 13 shows the binding of example CDH17xCD3 bispecific antibodies, h10G1tB and h10G4tB, to CD3 on Jurkat T cells;

FIG. 14 shows a safety feature of example CDH17xCD3 bispecific antibodies, h10G1fL, h10G4fL, h10G4tB, and h3G4tB that they do not activate T cells in the absence of tumor cells;

FIG. 15 demonstrates tumor cell dependent T cell activation by example CDH17xCD3 bispecific antibody, h10G4fL, using PBMC and AsPC1 tumor cells;

FIG. 16 shows example CDH17xCD3 bispecific antibody, h10G4fL, redirecting T cell cytotoxicity to CDH17 positive pancreatic and colon cancer cell lines in a concentration-dependent manner;

FIG. 17 shows the pharmacokinetics analysis of serum concentration of example CDH17xCD3 bispecific antibody, h10G4fL, following intravenous injection into: A) mice at 3 mg/kg (A) and B) a non-human primate (NHP) model at 3 mg/kg and 10 mg/kg;

FIG. 18 shows the histopathological analysis of example CDH17xCD3 bispecific antibody, h10G4fL, in NHP colon and pancreas from (A) necropsy samples and (B) in vivo model; and

FIG. 19 shows that the example CDH17xCD3 bispecific antibody, ARB202, can inhibit tumor growth in a mouse model of AsPC-1 cell-derived pancreatic cancer. A: Determination of tumor volume over 4 weeks of time in mice treated with RPMI (vehicle), PBMC-derived activated T cells (T cells), T cells plus 0.05 mg/kg ARB202, or T cells plus 0.5 mg/kg ARB202, at the indicated time points via intratumor administration; and B: Only the treatment with ARB202 resulted in increased levels of human IL-2 in plasma.

### DETAILED DESCRIPTION

The applications provide antibodies specific for both cadherin-17 (CDH17) and CD3, antibodies targeting tumor cells and anti-tumor immunotherapies using such antibodies. Such immunotherapies include antibodies possessing different modes of cytotoxicity or chimeric antigen receptors that stimulate T or NK cell cytotoxicity.

Unless defined otherwise, all technical and scientific terms used herein have the same meaning as commonly understood by those of ordinary skill in the art to which the present disclosure belongs. Although any methods and materials similar or equivalent to those described herein can be used in the practice or testing of the present disclosure, methods and materials are described. For the purposes of the present disclosure, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene.

Throughout this specification, unless the context requires otherwise, the words “comprise,” “comprises” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but those other elements are optional and may or may not be present depending upon whether or not they affect the activity or action of the listed elements.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

By “corresponds to” or “corresponding to” is meant (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

As used herein, the terms “function” and “functional” and the like refer to a biological, binding, or therapeutic function.

By “gene” is meant a unit of inheritance that occupies a specific locus on a chromosome and consists of transcriptional and/or translational regulatory sequences and/or a coding region and/or non-translated sequences (i.e., introns, 5′ and 3′ untranslated sequences).

“Homology” refers to the percentage number of amino acids that are identical or constitute conservative substitutions. Homology may be determined using sequence comparison programs such as GAP (Deveraux et al., 1984, Nucleic Acids Research 12, 387-395) which is incorporated herein by reference. In this way, sequences of a similar or substantially different length to those cited herein could be compared by insertion of gaps into the alignment, such gaps being determined, for example, by the comparison algorithm used by GAP.

The term “host cell” includes an individual cell or cell culture which can be or has been a recipient of any recombinant vector(s) or isolated polynucleotide of the present disclosure. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the present disclosure. A host cell which comprises a recombinant vector of the present disclosure is a recombinant host cell.

An “isolated” antibody is one which has been identified and separated and/or recovered from a component of its natural environment. Contaminant components of its natural environment are materials which would interfere with diagnostic or therapeutic uses for the antibody and may include enzymes, hormones, and other proteinaceous or non-proteinaceous solutes.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules, therefore, are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The recitation “polynucleotide” or “nucleic acid” as used herein designates mRNA, RNA, cRNA, rRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA and RNA.

The terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide, or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs that encode these enzymes.

“Polypeptide,” “polypeptide fragment,” “peptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally occurring amino acid polymers. In certain aspects, polypeptides may include enzymatic polypeptides, or “enzymes,” which typically catalyze (i.e., increase the rate of) various chemical reactions.

The recitation polypeptide “variant” refers to polypeptides that are distinguished from a reference polypeptide sequence by the addition, deletion or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions and, in this regard; it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues.

The term “reference sequence” generally refers to a nucleic acid coding sequence, or amino acid sequence, to which another sequence is being compared. All polypeptide and polynucleotide sequences described herein are included as references sequences.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 97%, 98%, 99% or 100% sequence identity to any of the reference sequences described herein (see, e.g., Sequence Listing), typically where the polypeptide variant maintains at least one biological activity of the reference polypeptide.

By “statistically significant,” it is meant that the result was unlikely to have occurred by chance. Statistical significance can be determined by any method known in the art. Commonly used measures of significance include the p-value, which is the frequency or probability with which the observed event would occur if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance, 95%, 96%, 97%, 98%, 99% or greater of some given quantity.

“Treating” or “treatment” or “alleviation” refers to both therapeutic treatment and prophylactic or preventative measures, wherein the object is to prevent or slow down (lessen) the targeted pathologic condition or disorder. For example, for cancer, reduction in the number of cancer cells or absence of the cancer cells; reduction in the tumor size; inhibition (i.e., slow to some extent and preferably stop) of tumor metastasis; inhibition, to some extent, of tumor growth; increase in length of remission, and/or relief to some extent, one or more of the symptoms associated with the specific cancer; reduced morbidity and mortality, and improvement in quality of life issues. Reduction of the signs or symptoms of a disease may also be felt by the patient. Treatment can achieve a complete response, defined as disappearance of all signs of cancer, or a partial response, wherein the size of the tumor is decreased, preferably by more than 50 percent, more preferably by 75%. A patient is also considered treated if the patient experiences stable disease. In one embodiment, the cancer patients are still progression-free in cancer after one year, preferably after 15 months. These parameters for assessing successful treatment and improvement in the disease are readily measurable by routine procedures familiar to a physician of appropriate skill in the art.

The terms “modulating” and “altering” include “increasing” and “enhancing” as well as “decreasing” or “reducing,” typically in a statistically significant or a physiologically significant amount or degree relative to a control. In specific embodiments, immunological rejection associated with transplantation of the blood substitutes is decreased relative to an unmodified or differently modified stem cell by at least 10%, at least 15%, at least 20%, at least 25%, at least 30%, at least 40%, at least 50%, at least 60%, at least 70%, at least 80%, at least 90%, at least 100%, at least 150%, at least 200%, at least 300%, at least 400%, at least 500%, or at least 1000%.

An “increased” or “enhanced” amount is typically a “statistically significant” amount, and may include an increase that is 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

A “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

By “obtained from” is meant that a sample such as, for example, a polynucleotide or polypeptide is isolated from, or derived from, a particular source, such as the desired organism or a specific tissue within the desired organism. “Obtained from” can also refer to the situation in which a polynucleotide or polypeptide sequence is isolated from, or derived from, a particular organism or tissue within an organism. For example, a polynucleotide sequence encoding a reference polypeptide described herein may be isolated from a variety of prokaryotic or eukaryotic organisms, or from particular tissues or cells within a certain eukaryotic organism. A “therapeutically effective amount” refers to an amount of an antibody or a drug effective to “treat” a disease or disorder in a subject. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See preceding definition of “treating.”

“Chronic” administration refers to administration of the agent(s) in a continuous mode as opposed to an acute mode, so as to maintain the initial therapeutic effect (activity) for an extended period of time. “Intermittent” administration is a treatment that is not consecutively done without interruption, but rather is cyclic in nature.

“Vector” includes shuttle and expression vectors. Typically, the plasmid construct will also include an origin of replication (e.g., the ColE1 origin of replication) and a selectable marker (e.g., ampicillin or tetracycline resistance), for replication and selection, respectively, of the plasmids in bacteria. An “expression vector” refers to a vector that contains the necessary control sequences or regulatory elements for expression of the antibodies including antibody fragment of the present disclosure, in bacterial or eukaryotic cells. Suitable vectors are disclosed below.

The term “antibody” is used in the broadest sense and specifically covers monoclonal antibodies (including full-length monoclonal antibodies), multispecific antibodies (e.g., bispecific antibodies), and antibody fragments so long as they exhibit the desired biological activity or function.

“Antibody fragments” comprise a portion of a full-length antibody, generally the antigen-binding or variable region of the antibody. Examples of antibody fragments include Fab, Fab′, F(ab′)2, and Fv fragments; diabodies; linear antibodies; single-chain antibody molecules; and multispecific antibodies formed from antibody fragments.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of a Fv comprising only three complementarity determining regions (CDRs) specific for an antigen) has the ability to recognize and bind antigen, although at a lower affinity than the entire binding site.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies and is not to be construed as requiring the production of the antibody by any particular method. For example, the monoclonal antibodies to be used in accordance with the present disclosure may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The term “variable” refers to the fact that certain segments of the variable domains (V domains) differ extensively in sequence among antibodies. The V domain mediates antigen binding and defines the specificity of a particular antibody for its particular antigen. However, the variability is not evenly distributed across the 10-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four frameworks regions (FRs), largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen but exhibit various effector functions, such as participation of the antibody in antibody-dependent cellular cytotoxicity (ADCC).

The term “hypervariable region” when used herein refers to the amino acid residues of an antibody which are responsible for antigen-binding. The hypervariable region generally comprises amino acid residues from a CDR (e.g. around about residues 24-34 (L1), 50-56 (L2) and 89-97 (L3) in the VL, and around about 31-35B (H1), 50-65 (H2) and 95-102 (H3) in the VH (Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the VL, and 26-32 (H1), 52A-55 (H2) and 96- 101 (H3) in the VH (Chothia and Lesk J. Mol. Biol. 196:901-917 (1987)).

“Chimeric” antibodies (immunoglobulins) have a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al. Proc. Natl Acad. Sci. USA 81:6851-6855 (1984)). Humanized antibody as used herein is a subset of chimeric antibodies.

“Humanized” forms of non-human (e.g., murine) antibodies are chimeric antibodies which contain minimal sequence derived from non-human immunoglobulin. In some embodiments, humanized antibodies are human immunoglobulins (recipient or acceptor antibody) in which hypervariable region residues of the recipient are replaced by hypervariable region residues from a non-human species (donor antibody) such as a mouse, rat, rabbit or nonhuman primate having the desired specificity, affinity, and capacity. In some embodiments, humanized antibodies are antibodies derived from human cells or from transgenic animals (typically mice) with express human antibody genes.

In one aspect, provided herein are antibodies or antigen-binding fragments thereof having specificity for CDH17. Tumor-associated antigens may serve as targets for anti-tumor immunotherapies by inhibiting their tumor growth promoting activities and by directing cytotoxic activity to tumor cells. CDH17 is a Type-1 integral transmembrane glycoprotein that belongs to the cadherin superfamily of cell adhesion molecules. It is a non-classical cadherin possessing 7 cadherin or cadherin-like repeats in its ectodomain. CDH17 is a tumor-associated antigen that participates in tumor growth. CDH17 expression normally restricted to intestinal epithelial cells of colon, small intestine, and pancreatic ducts are over-expressed in several tumors types including colon adenocarcinoma, gastric adenocarcinoma, hepatocellular carcinoma, cholangiocarcinoma, esophageal adenocarcinoma and pancreatic adenocarcinoma. Tumor growth promoting activity may involve binding between the RGD motif in CDH17 domain 6 and integrins such as α₂β₁. An abnormal increase in CDH17 level in blood and in exosomes may serve as prognostic cancer markers.

Using proteomics and oncogenomics approaches and through extensive research, a therapeutic target, liver-intestine cadherin or CDH17 is herein disclosed. The target is overexpressed in a majority of gastric carcinoma (GC) and hepatocellular carcinoma (HCC) as well as in pancreas cancer (panCA), colon cancer (CRC), ovary cancer and lung cancers. RNAi silencing of CDH17 gene could inhibit tumor growth and metastatic spread in the established HCC mouse models (both xenograft and orthotopic). The underlying antitumor mechanism is based on inactivation of Wnt signaling in concomitance with tumor suppressor pathway reactivation.

The anti-CDH17 antibodies present in this application have shown antitumor effects in multiple in vitro and in vivo systems of liver cancer and stomach cancers. Such antibodies have in vitro and in vivo purification, detection, diagnostic and therapeutic uses. Such antibodies may be developed to support anti-tumor activity by binding selectively to tumor cells and stimulate complement fixation, antibody-dependent cytotoxicity, cytotoxicity mediated by a conjugated drug, lymphocyte mediated cytotoxicity and NK-mediated cytotoxicity. Provided herein are antibodies and humanized antibodies, antigen-binding fragments or chimeric antibody proteins, comprising a heavy chain variable region having an amino acid sequence set forth as a corresponding SEQ ID provided below.

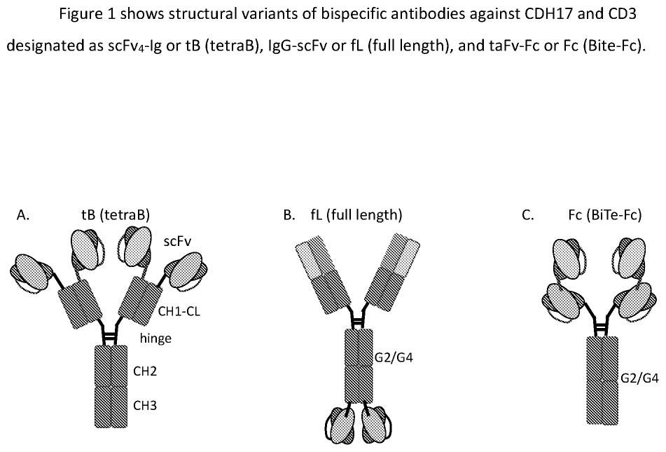

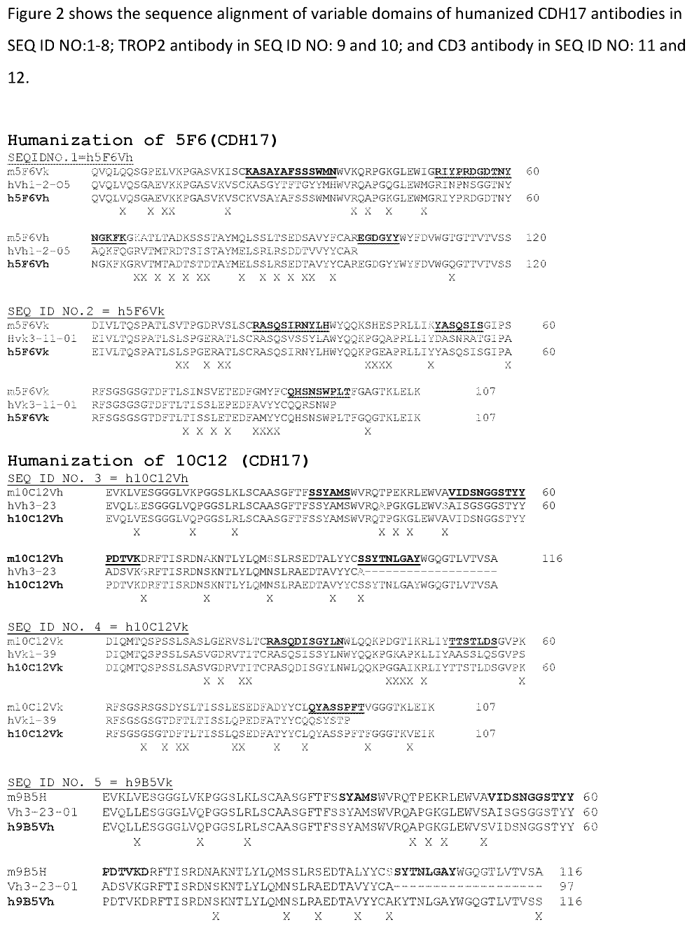

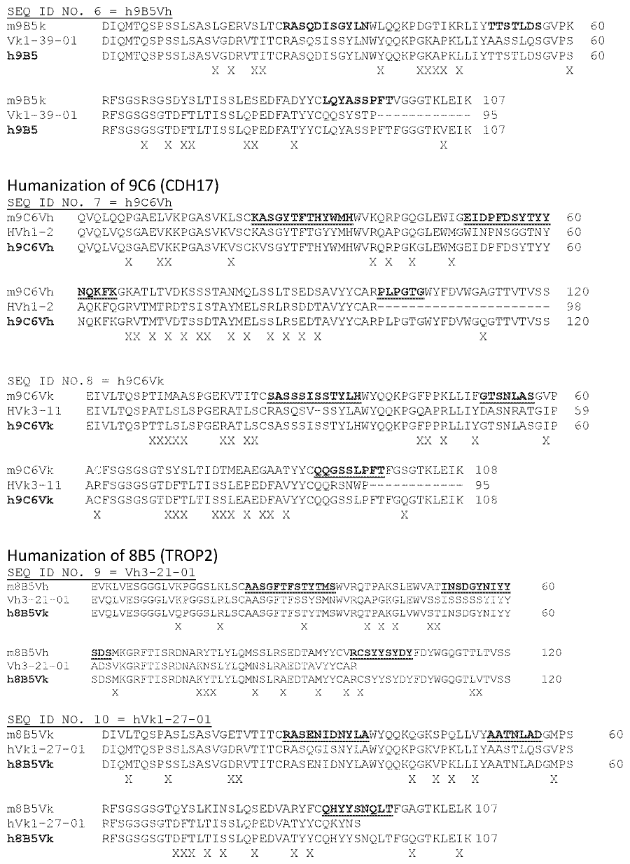

CDH17 antibody sequences may include various type of antibodies, such as mouse antibodies (5F6, 9B5, 9C6, 10C12, 8B5) and their humanized variants (FIGS. 1 and 2), i.e. bispecific antibodies, including various engineered antibody fragments (Fab, scFv, diabodies etc.). Example forms include “tB”, “fL” and “Fc” (FIG. 1).

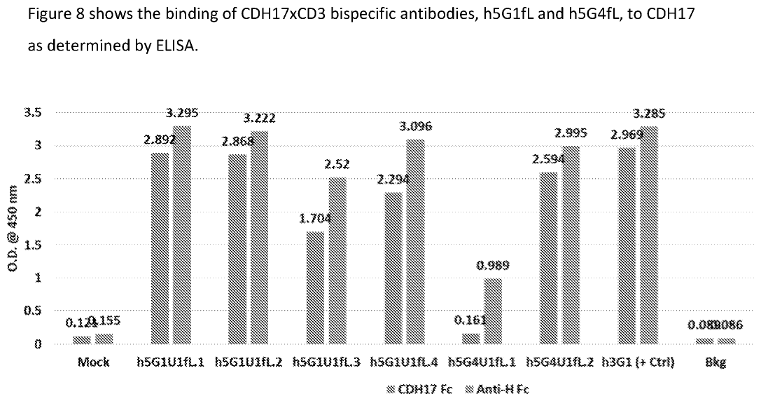

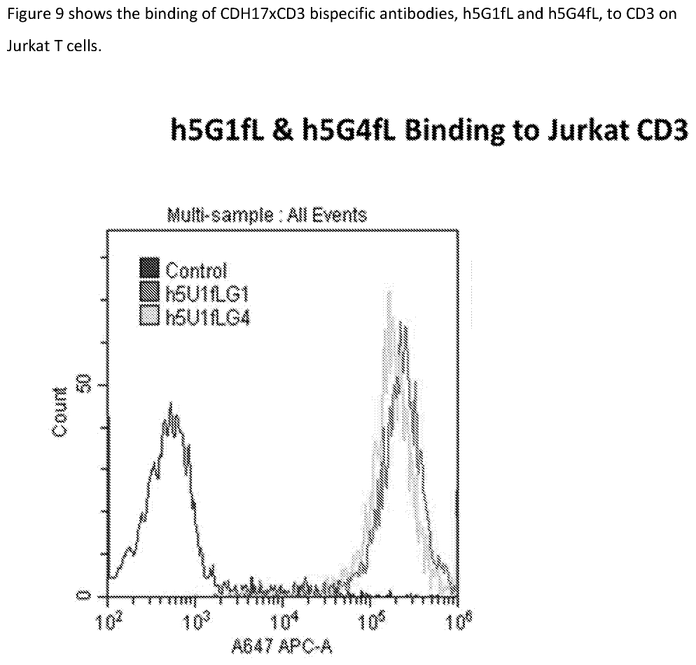

In some embodiments, humanized CDH17xCD3 bispecifc antibodies, h5G1fL, h5G4fL, h10G1fL, h10G4fL, h10G1tB and h5G4tB, display their ability to bind CDH17 in ELISA assays as shown in FIGS. 8, 10, and 12. Their ability to bind CD3 was demonstrated by flow cytofluorometry in FIGS. 9, 11, and 13.

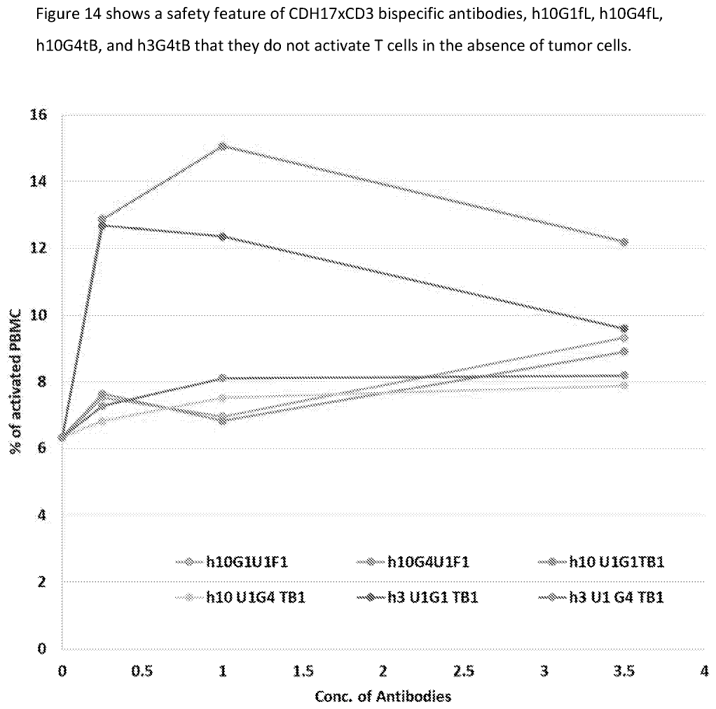

In another aspect, certain CDH17xCD3 bispecific antibodies, h10G1fL, h10G4fL, h10G4tB and h3G4tB display a safety feature in that they do not induce a cytotoxic T cell response when incubated with PBMCs in the absence of tumor cells (FIG. 14).

In one embodiment, antibodies may be identified that bind one of C-terminal ectodomains of CDH17, such as D5, D6 or D7. The binding may prevent CDH17 from being cleaved and shredded and may enable unique therapeutic activity of a novel mechanism. Such an anti-CDH17 antibody may be utilized in the construction of either a bispecific or a trispecific antibody that prevents CDH17 shedding while supporting T cell or NK killing of tumor cells. The second or third specificity of such an antibody may be CD3 or an NK cell receptor.

### EXAMPLES

The present disclosure is further described with reference to the following examples. These examples are provided for purposes of illustration only and are not intended to be limiting unless otherwise specified. Thus, the present disclosure should in no way be construed as being limited to the following examples, but rather, should be construed to encompass any and all variations which become evident as a result of the teaching provided herein.

### Example 1. Construction of CDH17xCD3 Bispecific Antibodies

CDH17xCD3 bispecific antibodies were generated and grouped based on their structural configuration: scFv₄-Ig or tB (tetraB), IgG-scFv or fL (full length), and taFv-Fc or Fc (Bite-Fc), as shown in FIG. 1 and listed in Table 1. All three types of designs contain U1, the scFv of a humanized UCHT-1 with its binding specificity to CD3. fL (full length) is a group of humanized anti-CDH17 antibody, whereas both tB (tetraB) and Fc (Bite-Fc) comprise an anti-CDH17 scFv and the scFv of humanized UCHT-1, respectively. Variable domains of CDH17 mouse antibodies, m5F6, m9B5, m9C6 and m10C12, and TROP2 mouse antibody m8B5 are aligned to homologous human germline sequences and humanized VH and VL sequences; h5F6, h9B5, h9C6, h10C12 and h8B5 as shown in FIG. 2. Humanized sequences include variants that may possess either a mouse or human germline residue at any position “X”. Variants may include a substitution at one or more positions. Variable domains of the anti-CD3 antibody UCHT-1, i.e. SEQ ID NO 11 and 12, were first humanized in 1992 (Beverley 1981 and Shalaby 1992). Amino acids at the sites designated with an “X” form hydrogen bond with CD3epsilon (Arnett 2004). Certain substitutions of these residues may result in decreased affinity for CD3.

### Example 2. Characterization of the h3/Fc Group of CDH17xCD3 Bispecific Antibodies

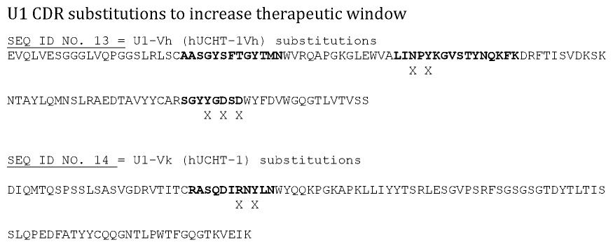

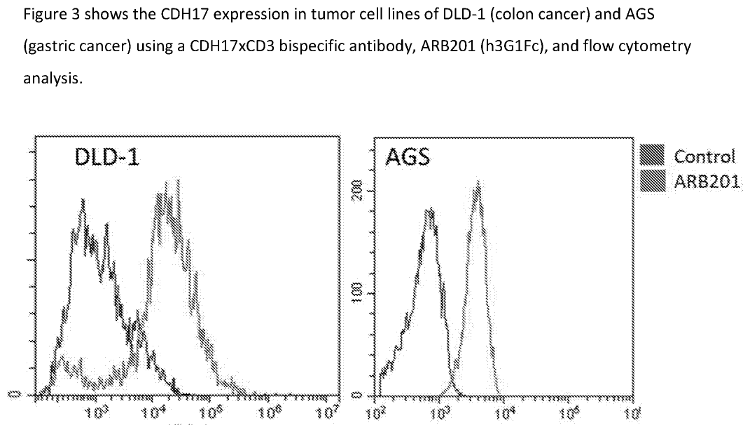

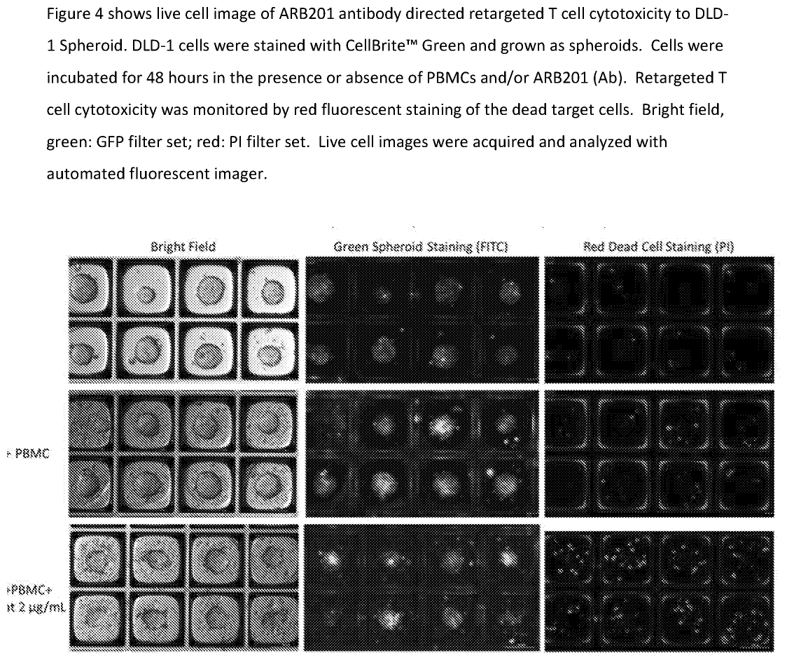

Of all CDH17xCD3 bispecific antibodies as listed in Table 1, h10G4fL was named as ARB202, and the monospecific version of ARB202 was named as ARB102. ARB201 is the same as h3G1Fc, which is not listed in Table 1 since the sequence for humanized variable domain h3 or Lic3 was disclosed in WO2017/120557A1. However, ARB201 was used to demonstrate that CDH17 is expression in tumor cell lines of DLD-1 (colon cancer) and AGS (gastric cancer) in a flow cytometry analysis (FIG. 3).

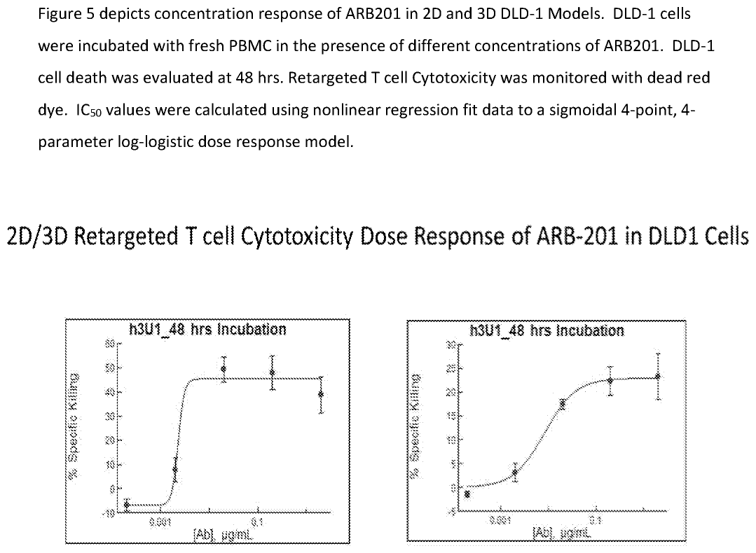

To determine whether ARB201 is sufficient to mediate retargeted T cell cytotoxicity to tumor cells, a standard 2-dimensional (2D) tumor cell and a 3-dimension (3D) tumor cell spheroid model. Colorectal cancer cells (DLD-1) expressing CDH17 were labeled with CellBrite™ Green (Biotium, Catalog No. 30021) and plated in microtiter wells with RPMI with % FCS. Peripheral blood mononuclear cells (PBMC) were isolated from healthy donors, separated by density-gradient centrifugation using Ficoll-Paque™ Plus (GE Healthcare), and used as effector cells. In the 3D model, tumors cells form spheroids after seeding in RPMI, 5% FBS, 2 mM L-alanyl-L-glutamine, 1 mM sodium pyruvate and 1% penicillin/streptomycin media culture in SQ 384-well Elplasia™ plates pre-coated with pHEMA hydrogel. In the assay, cells were incubated in the presence or absence of ARB201 for 16-48 hours until spheroids were formed. Dead cells were stained with a red fluorescent dye kit (EthD-III, Biotium, Catalog No. 30002) due to compromised plasma membranes. After one-hour, cellular analysis was conducted under a bright field, GFP and Texas Red® filter set, using a fluorescent imager. The IC₅₀ of ARB201 was 0.002 μg/mL in the 2D model and 0.008 μg/mL in the 3D model as shown in FIG. 5. The data indicates high potency in the 3D model (47pM) with only a 4-fold decrease relative to the 2D model. The 3D model may be more predictive than the 2D model for solid tumor cytotoxicity. Together these results indicate that ARB201 is capable of mediating retargeted T cell cytotoxicity to tumor cells.

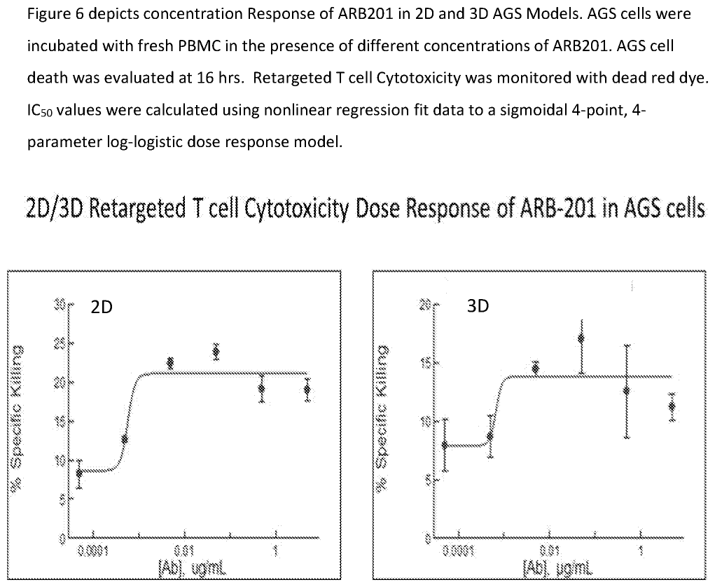

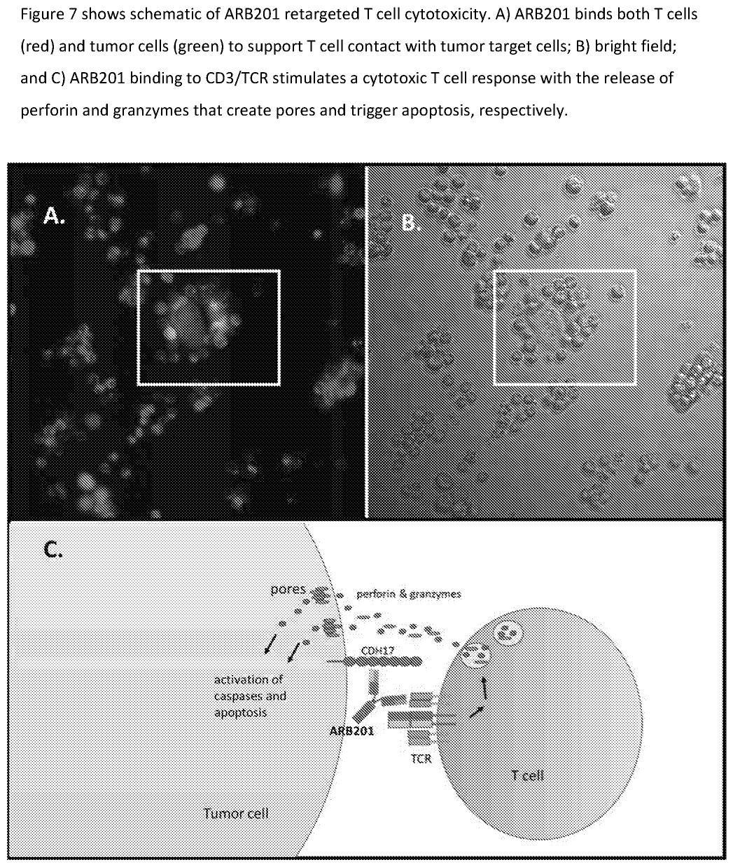

In addition to DLD-1 cell models, gastric cancer cells (AGS) were labeled with CellBrite™ Green and assayed in 2D and 3D tumor models as described for DLD-1 cells except the assay was measured at 16 hours. The IC₅₀ of ARB201 in these assays, as shown in FIG. 6, was 0.001 μg/mL in both the 2D and 3D models. The data indicates high potency with no decrease in potency in the 3D model relative to the 2D model. Moreover, in a live imaging study, the addition of ARB201 seemed to attract individual T cells and tumor cells together efficiently, as shown in FIG. 7. The finding supports that ARB201 stimulates a cytotoxic T cell response with the release of perforin and granzymes that create pores and trigger apoptosis, respectively.

Thus, the Fc group of CDH17xCD3 bispecific antibodies, ARB201, possesses high potency (low pM IC50) in both the 2D and 3D tumor models using CRC and GC tumor cells. There was no decrease in potency in the 3D model of GC and only a 4-fold decrease in the 3D model of CRC. The efficient killing in the 3D model may translate into clinical efficacy for solid tumours.

### Example 3. Characterization of the h5/fL Group of CDH17xCD3 Bispecific Antibodies

CDH17xCD3 bispecific antibodies,_h5G1fL and h5G4fL, were used to characterize the characters of the h5/fL group antibodies. To determine their binding specificity, CHO cells were used for expression and production of h5G1fL and h5G4fL, respectively. Different clones were incubated in the condition media in microtiter wells coated with recombinant CDH17 or anti-human IgG. Using ELISA. The binding of h5G1fL and h5G4fL to either CDH17 or anti-human IgG (to determine production) was detected using an anti-human Fc-HRP conjugate in ELISA. The relative binding activity can be measured and compared as shown in FIG. 8. The result indicates that the h5/fL group antibodies have their binding specificity to CDH17 comparable to the control anti-CDH17 antibody.

Next, h5G1fL and h5G4fL were incubated with Jurkat T cells, respectively. The binding was detected by subsequent binding of anti-human IgG Alexa647 conjugate in the flow cytofluorimetry analysis as shown in FIG. 9, indicating that anti-CD3 scFv is fully functional.

### Example 4. Characterization of the h10/fL Group of CDH17xCD3 Bispecific Antibodies

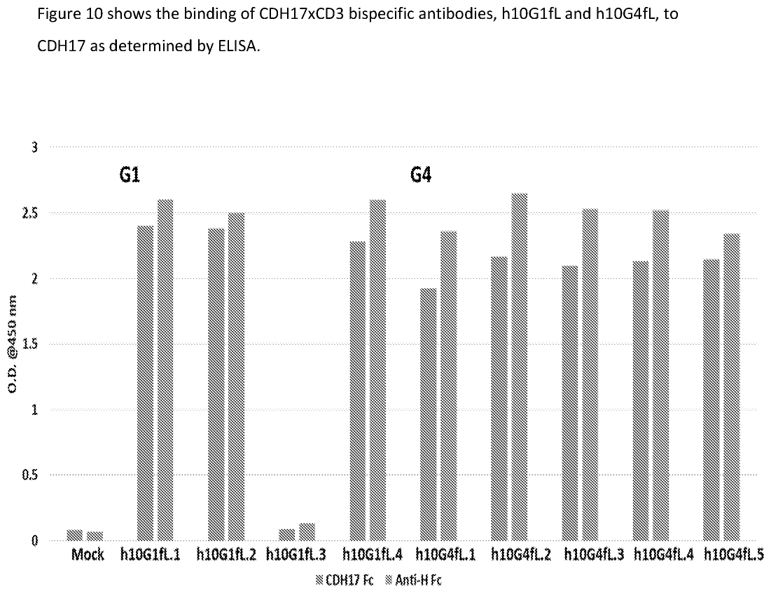

CDH17xCD3 bispecific antibodies, h10G1fL and h10G4fL, were used to characterize the characters of the h10/fL group antibodies. To determine their binding specificity, CHO cells were used for expression and production of h10G1fL and h10G4fL, respectively. Different clones were incubated in the condition media in microtiter wells coated with recombinant CDH17 or anti-human IgG. Using ELISA. The binding of h10G1fL and h10G4fL to either CDH17 or anti-human IgG (to determine production) was detected using an anti-human Fc-HRP conjugate in ELISA. The relative binding activity can be measured and compared as shown in FIG. 10. The result indicates that the h10/fL group antibodies have their binding specificity to CDH17 comparable up to the control anti-CDH17 antibody.

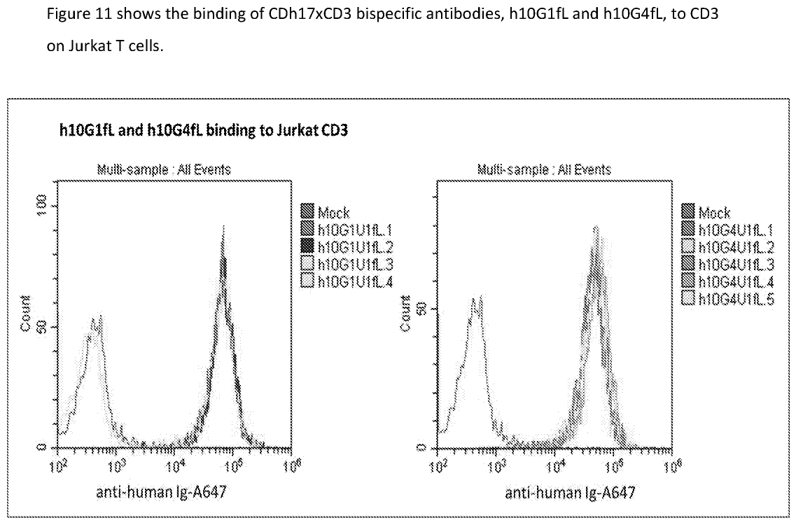

Next, h10G1fL and h10G4fL were incubated with Jurkat T cells, respectively. The binding was detected by subsequent binding of anti-human IgG Alexa647 conjugate in the flow cytofluorimetry analysis as shown in FIG. 11, indicating that anti-CD3 scFv is fully functional.

### Example 5. Characterization of the h10/tB Group of CDH17xCD3 Bispecific Antibodies

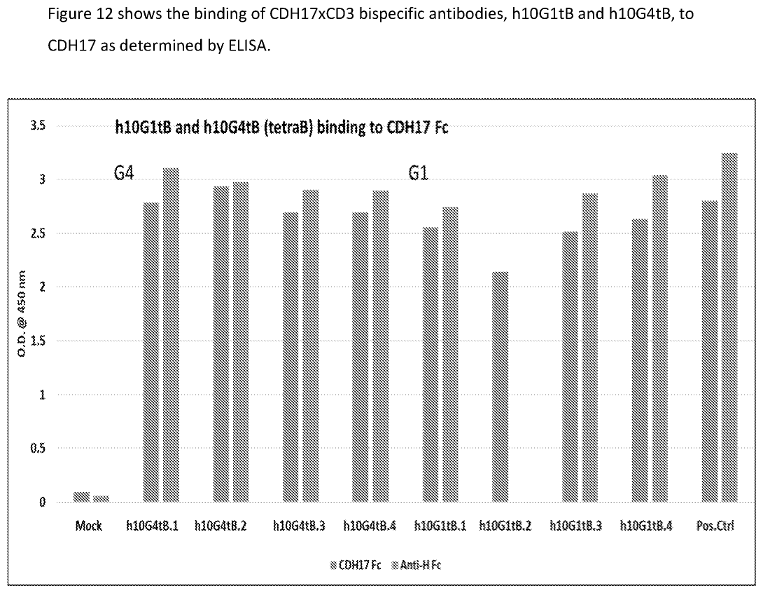

CDH17xCD3 bispecific antibodies, h10G1tB and h10G4tB, were used to characterize the characters of the h10/tB group antibodies. To determine their binding specificity, CHO cells were used for expression and production of h10G1tB and h10G4tB, respectively. Different clones were incubated in the condition media in microtiter wells coated with recombinant CDH17 or anti-human IgG. Using ELISA. The binding of h10G1tB and h10G4tB to either CDH17 or anti-human IgG (to determine production) was detected using an anti-human Fc-HRP conjugate in ELISA. The relative binding activity can be measured and compared as shown in FIG. 12. The result indicates that the h10/tB group antibodies have their binding specificity to CDH17 comparable up to the control anti-CDH17 antibody.

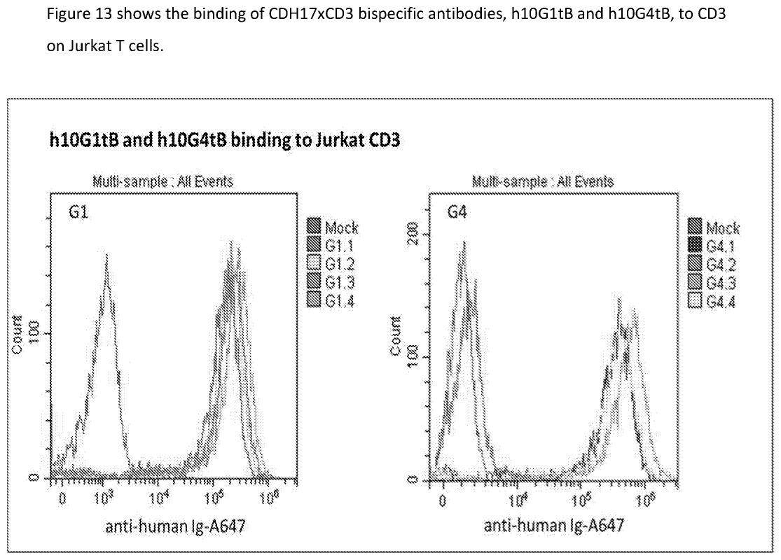

Next, h10G1fL and h5G4fL were incubated with Jurkat T cells, respectively. The binding was detected by subsequent binding of anti-human IgG Alexa647 conjugate in the flow cytofluorimetry analysis as shown in FIG. 13, indicating that anti-CD3 scFv is fully functional.

### Example 6. CDH17xCD3 Bispecific Antibodies with IgG4 Isotype do not Activate T Cells in the Absence of Tumor Cells

CDH17xCD3 bispecific antibodies, h10G1fL, h10G4fL, h10G4tB, and h3G4tB, were used in this analysis. Fresh PBMCs were incubated with each of four CDH17xCD3 bi-specific antibodies at a concentration range of 0-4 ug/ml in microtiter wells for 24 hours at 37° C. T cell activation and cytotoxic response were determined by staining cells with anti-CD107a antibody and anti-mlgG-fluorescent conjugate in flow cytofluorimetry. The percent of CD107a positive cells, indicative of cytotoxic T cell activation, were plotted versus antibody concentration. As shown in FIG. 14, the fL and IgG4 tB antibodies did not induce CD107a expression, and only tB with IgG1 isotype induced CD107a expression. The result reveals a safety feature in that the CDH17xCD3 bispecific antibodies of unique sub-structural configuration did not induce a cytotoxic T cell response as determined by CD107a expression when incubated with PBMCs in the absence of tumor cells.

### Example 7. The CDH17xCD3 Bispecific Antibody, h10G4fL, Mediates Tumor Cell Dependent T Cell Activation

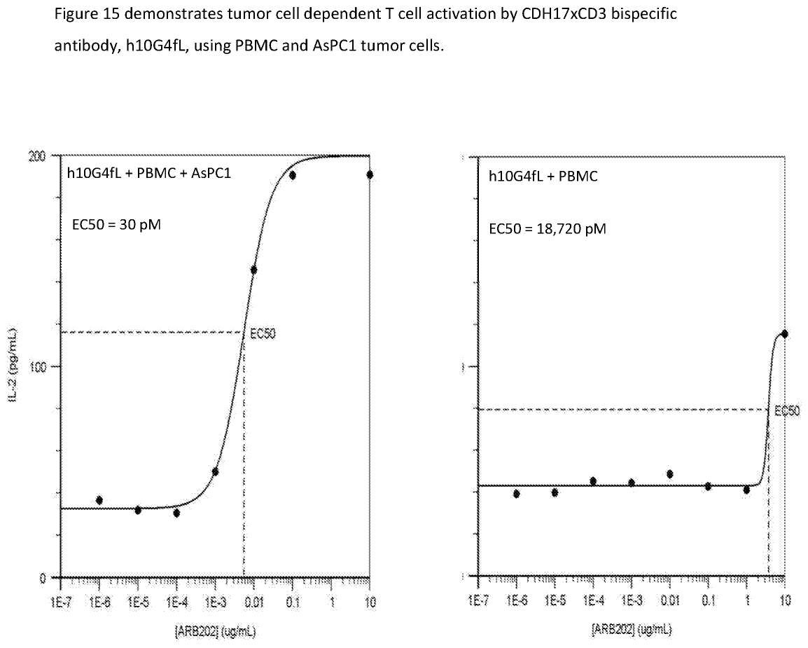

To characterize tumor cell dependent T cell activation by CDH17xCD3 bispecific antibodies, PBMCs and h10G4fL were incubated with or without the tumor cell line AsPC1 at a 5:1 ratio for 16 hours. T cell activation was determined by measuring IL2 production using a quantitative ELISA kit. As shown in FIG. 15, in the presence of AsPC1, IL2 was induced in a h10G4fL concentration-dependent manner with EC₅₀=30 pM (A), and in the absence of AsPC1, IL2 was induced with an EC50≥18,720 pM (B). Thus, this CDH17xCD3 bispecific antibody, h10G4fL, displayed a potential therapeutic index of greater than 600-fold.

### Example 8. h10G4fL Redirects T Cell Cytotoxicity to CDH17 Positive Tumor Cells

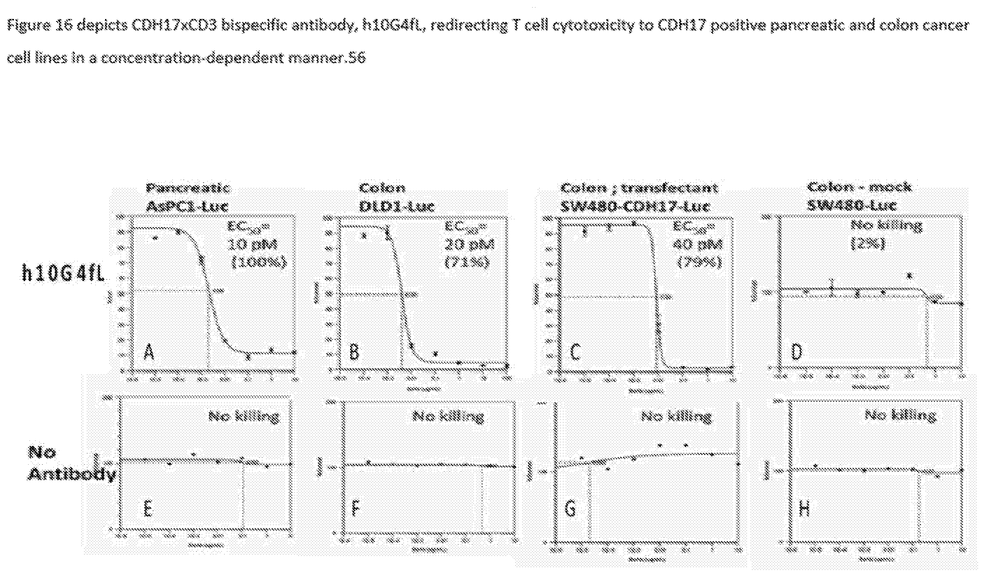

To further characterize the function of h10G4fL, both CDH17 positive and negative tumor cells were used to assess its ability in redirecting T cell cytotoxicity. Human PBMC-derived activated T cells and h10G4fL (or no antibody) were co-cultured with labelled tumor cells at a 5:1 ratio for 16 hours. At the end of incubation, each mixture was washed and the substrate was added to quantitate the remining viable cells and calculate the percent killing. T cell activation was determined by measuring IL2 production using a quantitative ELISA kit. As shown in FIG. 16, the result indicates that h10G4fL displayed concentration-dependent cytotoxicity to CDH17-positive luciferase labelled pancreatic and colon cell lines (FIGS. 16A and 16B), but not to CDH17 negative colon tumor cell line, SW40 (FIG. 16D). However, ectopic expression of CDH17 in SW40 conferred the sensitivity to h10G4fL dependent killing (FIG. 16C), demonstrating its specificity to target tumor cells.

### Example 9. Pharmacokinetic and Toxicology Analyses of h10G4fL/ARB202

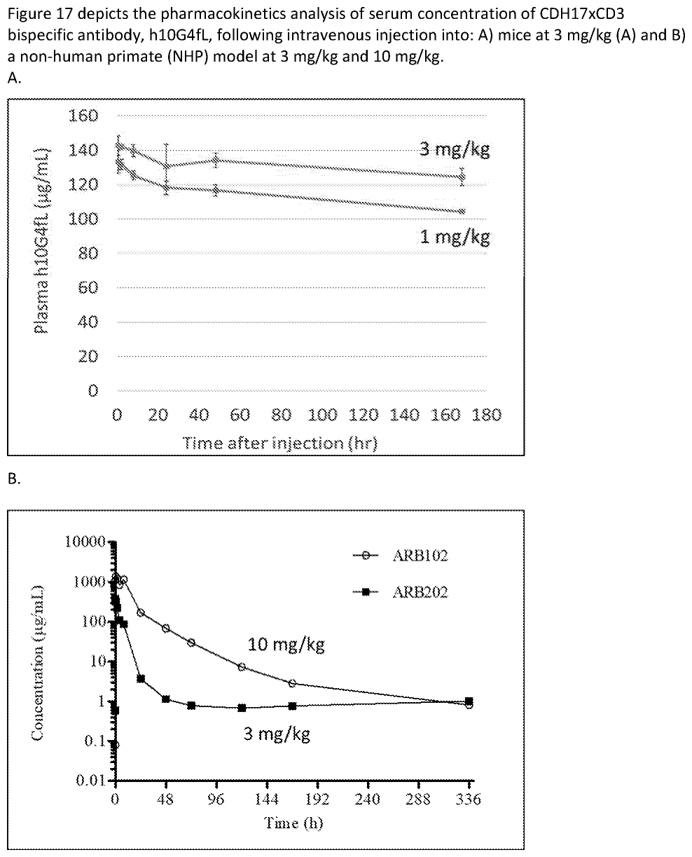

To determine the pharmacokinetics of h10G4fL, mice and non-human primates were used as small and large animal models. FIG. 17 displays the change of serum concentration of h10G4fL/ARB202 over time following intravenous injection at 3 mg/kg into mice (FIG. 17A) and a non-human primate (NHP) model (FIG. 17B). An isotype variant of h10G4fL/ARB202, h10G1/ARB102, was used for comparison at 1 mg/kg in mice (FIG. 17A) and 10 mg/kg in NHP (FIG. 17B).

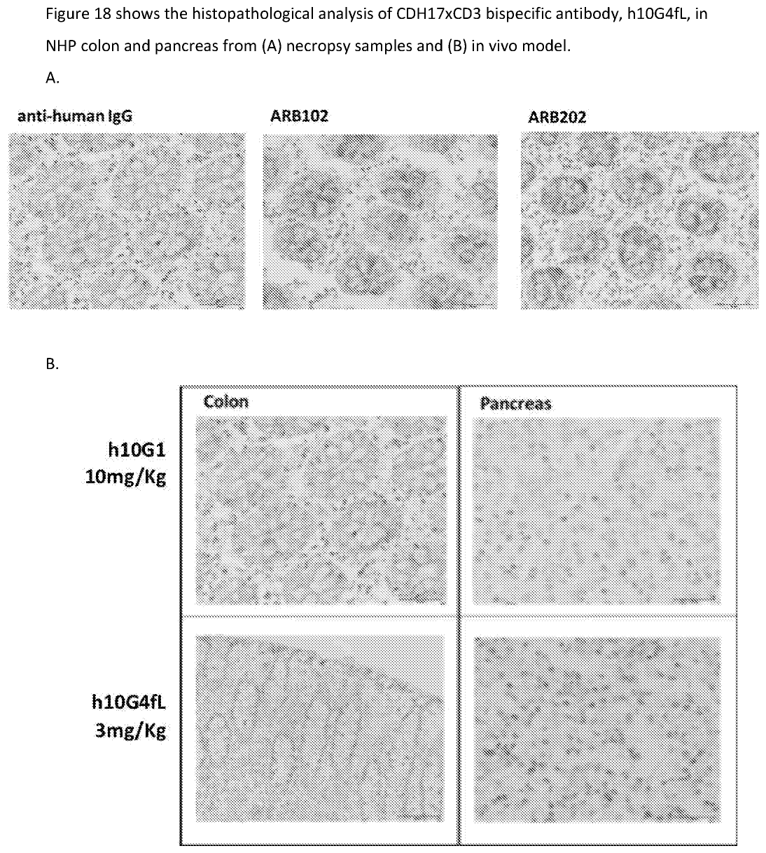

Then, both h10G4fL/ARB202 and h10G1/ARB102 were used in the preclinical cynomolgus monkey toxicity study at Charles River Laboratories. It was designed as a 14-day single dose study. It was previous determined that CDH17xCD3 bispecific antibodies can recognize and bind to cynomolgus CDH17 using cell transfectants. These antibodies also bind to monkey CDH17 in necropsy colon tissues as shown in immunohistochemistry (IHC) analysis. However, there was no evidence that these CDH17xCD3 bispecific antibodies can access and bind to CDH17 in the colon during the in-life phase. This issue was addressed by using post-necropsy colon tissue and anti-human IgG antibody in IHC analysis as shown in FIG. 18. In addition, there was no diarrhea nor dose dependent occult fecal blood, which could be indicators of inflammatory tissue damage associated with antibody treatment. Overall, the pathology report concluded that there was no morbidity and no gross or microscopic findings attributed to either ARB102 or ARB202 for the animals assigned to the study. The data supports the notion of safety that CDH17 or at least this epitope may not be accessible in normal colon and that therapeutic treatment may spare normal tissues.

### Example 10. The Efficacy Analysis of ARB202

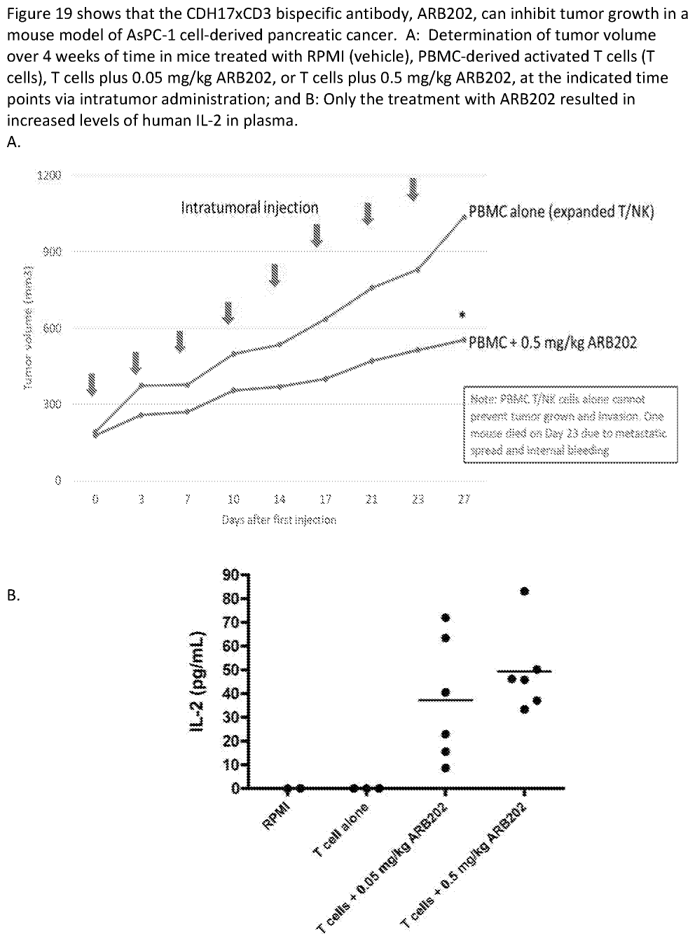

To determine the efficacy of ARB202 for treating CDH17 positive tumours in vivo, mouse xenograft models were used. A pancreas tumor model was established in NSB mice via subcutaneous injection of AsPC-1 pancreatic tumor cells. Mice were then treated via intratumor administration of vehicle (RPMI), T cells, T cells plus 0.05 mg/kg ARB202 or T cells plus 0.5 mg/kg ARB202 at each time point as indicated in FIG. 19. Tumor volume was determined over 4 weeks. The result shows that only low and high dose ARB202 resulted in a significant decrease in tumor growth relative to vehicle (P<0.05 comparing with RPMI injection). The statistically insignificant decrease in tumor growth observed with T cells alone was likely due to high NK activity in the expanded T cell population and NK sensitivity of AsPC-1. To further validate the T cell activation in the process, the serum IL-2 was analyzed. The result shows that only the treatment with ARB202 resulted in increased levels of human IL-2 in plasma. Thus, ARB202 provides a proof of concept that CDH17xCD3 bispecific antibodies can be used for treating CDh17 positive tumours.

### Pharmaceutical Compositions

The term “effective amount” refers to an amount of a drug effective to achieve a desired effect, e.g., to ameliorate disease in a subject. Where the disease is cancer, the effective amount of the drug may inhibit (for example, slow to some extent, inhibit or stop) one or more of the following example characteristics including, without limitation, cancer cell growth, cancer cell proliferation, cancer cell motility, cancer cell infiltration into peripheral organs, tumor metastasis, and tumor growth. Wherein the disease is cancer, the effective amount of the drug may alternatively do one or more of the following when administered to a subject: slow or stop tumor growth, reduce tumor size (for example, volume or mass), relieve to some extent one or more of the symptoms associated with the cancer, extend progression-free survival, result in an objective response (including, for example, a partial response or a complete response), and increase overall survival time. To the extent the drug may prevent growth and/or kill existing cancer cells, it is cytostatic and/or cytotoxic.

With respect to the formulation of suitable compositions for administration to a subject such as a human patient in need of treatment, the antibodies disclosed herein may be mixed or combined with pharmaceutically acceptable carriers known in the art dependent upon the chosen route of administration. There are no particular limitations to the modes of application of the antibodies disclosed herein, and the choice of suitable administration routes and suitable compositions are known in the art without undue experimentation.

Although many forms of administration are possible, an example administration form would be a solution for injection, in particular for intravenous or intra-arterial injection. Usually, a suitable pharmaceutical composition for injection may include pharmaceutically suitable carriers or excipients such as, without limitation, a buffer, a surfactant, or a stabilizer agent. Example buffers may include, without limitation, acetate, phosphate or citrate buffer. Example surfactants may include, without limitation, polysorbate. Example stabilizer may include, without limitation, human albumin.

Similarly, persons skilled in the art have the ability to determine the effective amount or concentration of the antibodies disclosed therein to effective treat a condition such as cancer. Other parameters such as the proportions of the various components of the pharmaceutical composition, the administration does and frequency may be obtained by a person skilled in the art without undue experimentation. For example, a suitable solution for injection may contain, without limitation, from about 1 to about 20, from about 1 to about 10 mg antibodies per ml. The example dose may be, without limitation, from about 0.1 to about 20, from about 1 to about 5 mg/Kg body weight. The example administration frequency could be, without limitation, once per day or three times per week.

While the present disclosure has been described with reference to particular embodiments or examples, it may be understood that the embodiments are illustrative and that the disclosure scope is not so limited. Alternative embodiments of the present disclosure may become apparent to those having ordinary skill in the art to which the present disclosure pertains. Such alternate embodiments are considered to be encompassed within the scope of the present disclosure. Accordingly, the scope of the present disclosure is defined by the appended claims and is supported by the foregoing description.

In summary, we describe a 2D/3D platform for the study of retargeted T cell cytotoxicity of ARB201, a bispecific antibody targeting CDH17 & CD3. ARB201 induced retargeted T cell cytotoxicity in the DLD-1 colorectal adenocarcinoma cells with an IC₅₀ of 0.002 μg/mL in the 2D model and 0.008 μg/mL in the 3D model. In AGS gastric adenocarcinoma cells ARB201 also induced retargeted T cell with an IC₅₀ of 0.001 μg/mL in both the 2D and 3D models. This study demonstrated that ARB201 efficiently and progressively killed tumor cells in a 3D model with nearly the same efficiency as in the 2D model. Efficient killing in the 3D model may translate into clinical efficacy for solid tumours.

While the disclosure has been described with reference to particular embodiments, it will be understood that the embodiments are illustrative and that the disclosure scope is not so limited. Alternative embodiments of the present disclosure will become apparent to those having ordinary skill in the art to which the present disclosure pertains. Such alternate embodiments are considered to be encompassed within the scope of the present disclosure. Accordingly, the scope of the present disclosure is defined by the appended claims and is supported by the foregoing description.

The embodiments are merely for illustrating the present disclosure and are not intended to limit the scope of the present disclosure. It should be understood for persons in the technical field that certain modifications and improvements may be made and should be considered under the protection of the present disclosure without departing from the principles of the present disclosure.

### TABLES

TABLE 1
Design of Bispecific Antibodies Specific for CDH17 and CD3

| | Humanized variable domain | | | | | | | | |
|---|---|---|---|---|---|---|---|---|---|
| | scFV₄-Fc | | | IgG-scFv | | | taFv-Fc | | |
| | tB (tetraB) | | | fL (full length) | | | Fc (BiTE-Fc) | | |
| Isotype | G1 | G2 | G4 | G1 | G2 | G4 | G1 | G2 | G4 |
| h5 | h5G1tB | h5G2tB | h5G4tB | h5G1fL | h5G2fL | h5G4fL | h5G1Fc | h5G2Fc | h5G4Fc |
| (m5F6) | | | | | | | | | |
| h9B | h9BG1tB | h9BG2tB | h9BG4tB | h9BG1fL | h9BG2fL | h9BG4fL | h9BG1Fc | h9BG2Fc | h9BG4Fc |
| (m9B5) | | | | | | | | | |
| h9C | h9CG1tB | h9CG2tB | h9CG4tB | h9CG1fL | h9CG2fL | h9CG4fL | h9CG1Fc | h9CG2Fc | h9CG4Fc |
| (m9C6) | | | | | | | | | |
| h10 | h10G1tB | h10G2tB | h10G4tB | h10G1fL | h10G2fL | h10G4fL | h10G1Fc | h10G2Fc | h10G4Fc |
| (m10C12) | | | | | | ARB202 | | | |

TABLE 2
Examples of binding targets of multi-specific antibodies

| | Binding | | Binding |
|---|---|---|---|
| Antibody | Specificity 1 | Binding Specificity 2 | Specificity 3 |
| Bi-specific | CDH17 (D1-D7) | CD3, TROP2, GPC3, | N/A |
| | | HER2, CDH17 (D1-D7) | |
| Bi-specific | GPC3, HER2 | TROP2 | N/A |
| Tri-Specific | CDH17 (D1-D7) | TROP2, GPC3, HER2 | CD3 |

TABLE 3
Summary of fecal occult blood from NHP

| | Day −7 | Day 1 | Day 2 | Day 3 | Day 8 | Day 15 |
|---|---|---|---|---|---|---|
| ARB102 | | | | | | |
| 0.5 mg/kg | Neg | Neg | Neg | Neg | Neg | Neg |
| 10 mg/kg | Neg | Neg | Neg | Neg | Neg | Neg |
| ARB202 | | | | | | |
| 0.03 mg/kg | Neg | Neg | Pos* | Neg | Neg | Neg |
| 0.3 mg/kg | Neg | Neg | Neg | Neg | Neg | Neg |
| 3 mg/kg | Neg | Neg | Neg | Neg | Neg | Neg |
| | (day −11) | | | | | |

*Analysis of ARB202 levels in serum indicates that the dosing was correct.

SEQUENCE LISTING
Examples of CDH17xCD3 bispecific antibodies
Humanized amino acid sequences of 5F6 (CDH17) variable heavy domain
SEQ ID NO: 1
QVQLVQSGAEVKKPGASVKVSCKVSAYAFSSSWMNWVRQAPGKGLEWMGRIYPRDGDTNYNGKFKGRV
TMTADTSTDTAYMELSSLRSEDTAVYYCAREGDGYYWYFDVWGQGTTVTVSS
Humanized amino acid sequences of 5F6 (CDH17) variable light domain
SEQ ID NO: 2
EIVLTQSPATLSLSPGERATLSCRASQSIRNYLHWYQQKPGEAPRLLIYYASQSISGIPARFSGSGSGTDFTLTISS
LETEDFAMYYCQHSNSWPLTFGQGTKLEIK
Humanized amino acid sequences of 10C12 (CDH17) variable heavy domain
SEQ ID NO: 3
EVOLVESGGGLVQPGGSLRLSCAASGFTFSSYAMSWVRQTPGKGLEWVAVIDSNGGSTYYPDTVKDRFTISRDNSKNT
LYLQMNSLRAEDTAVYYCSSYTNLGAYWGQGTLVTVSA
Humanized amino acid sequences of 10C12 (CDH17) variable light domain
SEQ ID NO: 4
DIQMTQSPSSLSASVGDRVTITCRASQDISGYLNWLQQKPGGAIKRLIYTTSTLDSGVPKRFSGSGSGTDFTLTISS
LQSEDFATYYCLQYASSPFTFGGGTKVEIK
Humanized amino acid sequences of 9B5 (CDH17) variable heavy domain
SEQ ID NO: 5
EVQLLESGGGLVQPGGSLRLSCAASGFTFSSYAMSWVRQAPGKGLEWVSVIDSNGGSTYYPDTVKDRFTISR
DNSKNTLYLQMNSLRAEDTAVYYCAKYTNLGAYWGQGTLVTVSS
Humanized amino acid sequences of 9B5 (CDH17) variable light domain
SEQ ID NO: 6
DIQMTQSPSSLSASVGDRVTITCRASQDISGYLNWYQQKPGKAPKLLIYTTSTLDSGVPSRFSGSGSGTDFTLTI
SSLQPEDFATYYCLQYASSPFTFGGGTKVEIK
Humanized amino acid sequences of 9C6 (CDH17) variable heavy domain
SEQ ID NO: 7
QVQLVQSGAEVKKPGASVKVSCKVSGYTFTHYWMHWVRQRPGKGLEWMGEIDPFDSYTYYNQKFKGRVT
MTVDTSSDTAYMELSSLRSEDTAVYYCARPLPGTGWYFDVWGQGTTVTVSS
Humanized amino acid sequences of 9C6 (CDH17) variable light domain
SEQ ID NO: 8
EIVLTQSPTTLSLSPGERATLSCSASSSISSTYLHWYQQKPGFPPRLLIYGTSNLASGIPACFSGSGSGTDFTLTISS
LEAEDFAVYYCQQGSSLPFTFGQGTKLEIK
Humanized amino acid sequences of 8B5 (TROP2) variable heavy domain
SEQ ID NO: 9
EVQLVESGGGLVQPGGSLRLSCAASGFTFSTYTMSWVRQTPAKGLVWVSTINSDGYNIYYSDSMKGRFTISR
DNAKYTLYLQMNSLRAEDTAMYYCARCSYYSYDYFDYWGQGTLVTVSS
Humanized amino acid sequences of 8B5 (TROP2) variable light domain
SEQ ID NO: 10
DIQMTQSPSSLSASVGDRVTITCRASENIDNYLAWYQQKQGKVPKLLIYAATNLADGMPSRFSGSGSGTDFT
LTISSLQPEDVATYYCQHYYSNQLTFGQGTKLEIK
Humanized amino acid sequences of 3A4 (TROP2) variable heavy domain
SEQ ID NO: 11
QVQLVQSGAEVKKPGASVKVSCKASGYTFTDFYMNWVRQAPGQGLEWMGRVNPSNGDTNYNQKFKGR
VTSTRDTSISTAYMELSRLRSDDTAVYYCARERIYYGISWYFDVWDTGTTVTVSS
Humanized amino acid sequences of 3A4 (TROP2) variable light domain
SEQ ID NO: 12
DIQMTQhSPSSLSASVGDRVTITCRASGNIHNYLAWYQQKPGKAPKLLLYNAKTLAEGVPSRFSGSGSGTDYT
LTISSLQPEDFATYYCHHYYSTPPTFGQGTKLEIK
Examples of TetraB design bispecific antibodies
U1G1tB-common heavy chain for G1 tetraB bispecific antibodies
SEQ. ID: 13
MEFGLSWVFLVALLRGVQCEVQLVESGGGLVQPGGSLRLSCAASGYSFTGYTMNWVRQAPGKGLEWVALI
NPYKGVSTYNQKFKDRFTISVDKSKNTAYLQMNSLRAEDTAVYYCARSGYYGDSDWYFDVWGQGTLVTVSS
GGGGSGGGGSGGGGSDIQMTQSPSSLSASVGDRVTITCRASQDIRNYLNWYQQKPGKAPKLLIYYTSRLESG
VPSRFSGSGSGTDYTLTISSLQPEDFATYYCQQGNTLPWTFGQGTKVEIKPAPAPASTKGPSVFPLAPSSKSTS
GGTAALGCLVKDYFPEPVTVSWNSGALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVDKSRWQQGNVFSCS
VMHEALHNHYTQKSLSLSPGK
U1G2tB-common heavy chain for G2 tetraB bispecific antibodies
SEQ. ID: 14
MEFGLSWVFLVALLRGVQCEVQLVESGGGLVQPGGSLRLSCAASGYSFTGYTMNWVRQAPGKGLEWVALI
NPYKGVSTYNQKFKDRFTISVDKSKNTAYLQMNSLRAEDTAVYYCARSGYYGDSDWYFDVWGQGTLVTVSS
GGGGSGGGGSGGGGSDIQMTQSPSSLSASVGDRVTITCRASQDIRNYLNWYQQKPGKAPKLLIYYTSRLESG
VPSRFSGSGSGTDYTLTISSLQPEDFATYYCQQGNTLPWTFGQGTKVEIKPAPAPASTKGPSVFPLAPCSRSTS
ESTAALGCLVKDYFPEPVTVSWNSGALTSGVHTFPAVLQSSGLYSLSSVVTVPSSNFGTQTYTCNVDHKPSNT
KVDKTVERKCCVECPPCPAPPVAGPSVFLFPPKPKDTLMISRTPEVTCVVVDVSHEDPEVQFNWYVDGVEVH
NAKTKPREEQFNSTFRVVSVLTVVHQDWLNGKEYKCKVSNKGLPAPIEKTISKTKGQPREPQVYTLPPSREEM
TKNQVSLTCLVKGFYPSDISVEWESNGQPENNYKTTPPMLDSDGSFFLYSKLTVDKSRWQQGNVFSCSVMH
EALHNHYTQKSLSLSPGK
U1G4tB-common heavy chain for G4 tetraB bispecific antibodies
SEQ. ID: 15
MEFGLSWVFLVALLRGVQCEVQLVESGGGLVQPGGSLRLSCAASGYSFTGYTMNWVRQAPGKGLEWVALI
NPYKGVSTYNQKFKDRFTISVDKSKNTAYLQMNSLRAEDTAVYYCARSGYYGDSDWYFDVWGQGTLVTVSS
GGGGSGGGGSGGGGSDIQMTQSPSSLSASVGDRVTITCRASQDIRNYLNWYQQKPGKAPKLLIYYTSRLESG
VPSRFSGSGSGTDYTLTISSLQPEDFATYYCQQGNTLPWTFGQGTKVEIKPAPAPASTKGPSVFPLAPSSKSTS
GGTAALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPELLGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSFFLYSRLTVDKSRWQEGNVFSCS
VMHEALHNHYTQKSLSLSLGK
h5KtB light chain
SEQ. ID: 16
MEFGLSWVFLVALLRGVQCQVQLVQSGAEVKKPGASVKVSCKVSAYAFSSSWMNWVRQAPGKGLEWM
GRIYPRDGDTNYNGKFKGRVTMTADTSTDTAYMELSSLRSEDTAVYYCAREGDGYYWYFDVWGQGTTVTV
SSGGGGSGGGGSGGGGSEIVLTQSPATLSLSPGERATLSCRASQSIRNYLHWYQQKPGEAPRLLIYYASQSISG
IPARFSGSGSGTDFTLTISSLETEDFAMYYCQHSNSWPLTFGQGTKLEIKPAGGGGSGRTVAAPSVFIFPPSDE
QLKSGTASVVCLLNNFYPREAKVQWKVDNALQSGNSQESVTEQDSKDSTYSLSSTLTLSKADYEKHKVYACE
VTHQGLSSPVTKSFNRGEC
h10KtB light chain
SEQ. ID: 17
MEFGLSWVFLVALLRGVQCEVQLVESGGGLVQPGGSLRLSCAASGFTFSSYAMSWVRQTPGKGLEWVAVI
DSNGGSTYYPDTVKDRFTISRDNSKNTLYLQMNSLRAEDTAVYYCSSYTNLGAYWGQGTLVTVSAGGGGSG
GGGSGGGGSDIQMTQSPSSLSASVGDRVTITCRASQDISGYLNWLQQKPGGAIKRLIYTTSTLDSGVPKRF
SGSGSGTDFTLTISSLQSEDFATYYCLQYASSPFTFGGGTKVEIKPAGGGGSGRTVAAPSVFIFPPSDE
QLKSGTASVVCLLNNFYPREAKVQWKVDNALQSGNSQESVTEQDSKDSTYSLSSTLTLSKADYEKHKVYA
CEVTHQGLSSPVTKSFNRGEC
Examples of fL design bispecific antibodies
heavy chain for h10G1fL (h10G1U1fL)
SEQ. ID: 18
MEFGLSWVFLVALLRGVQCEVQLVESGGGLVQPGGSLRLSCAASGFTFSSYAMSWVRQTPGKGLEWVAVI
DSNGGSTYYPDTVKDRFTISRDNSKNTLYLQMNSLRAEDTAVYYCSSYTNLGAYWGQGTLVTVSAASTKGPS
VFPLAPSSKSTSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSDGSFFLYSKLTVDKSR
WQQGNVFSCSVMHEALHNHYTQKSLSLSPGKPAGGGGSEVQLVESGGGLVQPGGSLRLSCAASGYSFTGY
TMNWVRQAPGKGLEWVALINPYKGVSTYNQKFKDRFTISVDKSKNTAYLQMNSLRAEDTAVYYCARSGYY
GDSDWYFDVWGQGTLVTVSSGGGGSGGGGSGGGGSDIQMTQSPSSLSASVGDRVTITCRASQDIRNYLN
WYQQKPGKAPKLLIYYTSRLESGVPSRFSGSGSGTDYTLTISSLQPEDFATYYCQQGNTLPWTFGQGTKVEIK
heavy chain for h10G2fL (h10G2U1fL)
SEQ. ID: 19
MEFGLSWVFLVALLRGVQCEVQLVESGGGLVQPGGSLRLSCAASGFTFSSYAMSWVRQTPGKGLEWVAVI
DSNGGSTYYPDTVKDRFTISRDNSKNTLYLQMNSLRAEDTAVYYCSSYTNLGAYWGQGTLVTVSSASTKGPS
VFPLAPCSRSTSESTAALGCLVKDYFPEPVTVSWNSGALTSGVHTFPAVLQSSGLYSLSSVVTVPSSNFGTQTY
TCNVDHKPSNTKVDKTVERKCCVECPPCPAPPVAGPSVFLFPPKPKDTLMISRTPEVTCVVVDVSHEDPEVQ
FNWYVDGVEVHNAKTKPREEQFNSTFRVVSVLTVVHQDWLNGKEYKCKVSNKGLPAPIEKTISKTKGQPRE
PQVYTLPPSREEMTKNQVSLTCLVKGFYPSDISVEWESNGQPENNYKTTPPMLDSDGSFFLYSKLTVDKSRW
QQGNVFSCSVMHEALHNHYTQKSLSLSPGKPAGGGGSEVQLVESGGGLVQPGGSLRLSCAASGYSFTGYT
MNWVRQAPGKGLEWVALINPYKGVSTYNQKFKDRFTISVDKSKNTAYLQMNSLRAEDTAVYYCARSGYYG
DSDWYFDVWGQGTLVTVSSGGGGSGGGGSGGGGSDIQMTQSPSSLSASVGDRVTITCRASQDIRNYLNW
YQQKPGKAPKLLIYYTSRLESGVPSRFSGSGSGTDYTLTISSLQPEDFATYYCQQGNTLPWTFGQGTKVEIK
heavy chain for h10G4fL (h10G4U1fL)
SEQ. ID: 20
MEFGLSWVFLVALLRGVQCEVQLVESGGGLVQPGGSLRLSCAASGFTFSSYAMSWVRQTPGKGLEWVAVI
DSNGGSTYYPDTVKDRFTISRDNSKNTLYLQMNSLRAEDTAVYYCSSYTNLGAYWGQGTLVTVSSASTKGPS
VFPLAPSSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSQEDPE
VQFNWYVDGVEVHNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDGSFFLYSRLTVDKSR
WQEGNVFSCSVMHEALHNHYTQKSLSLSLGKPAGGGGSEVQLVESGGGLVQPGGSLRLSCAASGYSFTGYT
MNWVRQAPGKGLEWVALINPYKGVSTYNQKFKDRFTISVDKSKNTAYLQMNSLRAEDTAVYYCARSGYYG
DSDWYFDVWGQGTLVTVSSGGGGSGGGGSGGGGSDIQMTQSPSSLSASVGDRVTITCRASQDIRNYLNW
YQQKPGKAPKLLIYYTSRLESGVPSRFSGSGSGTDYTLTISSLQPEDFATYYCQQGNTLPWTFGQGTKVEIK
Light chain for all h10 fL (h10Kappa)
SEQ. ID: 21
MRLPAQLLGLLMLWVSGSSGDIQMTQSPSSLSASVGDRVTITCRASQDISGYLNWLQQKPGGAIKRLIYTTS
TLDSGVPKRFSGSGSGTDFTLTISSLQSEDFATYYCLQYASSPFTFGGGTKVEIKRTVAAPSVFIFPPSDEQLKSG
TASVVCLLNNFYPREAKVQWKVDNALQSGNSQESVTEQDSKDSTYSLSSTLTLSKADYEKHKVYACEVTHQG
LSSPVTKSFNRGEC
Examples of Fc (Bite-Fc) design bispecific antibodies
single chain (heavy) for h10G1 (h10U1G1)
SEQ. ID: 22
MEFGLSWVFLVALLRGVQCEVQLVESGGGLVQPGGSLRLSCAASGFTFSSYAMSWVRQTPGKGLEWVAVI
DSNGGSTYYPDTVKDRFTISRDNSKNTLYLQMNSLRAEDTAVYYCSSYTNLGAYWGQGTLVTVSAGGGGSG
GGGSGGGGSDIQMTQSPSSLSASVGDRVTITCRASQDISGYLNWLQQKPGGAIKRLIYTTSTLDSGVPKRFSG
SGSGTDFTLTISSLQSEDFATYYCLQYASSPFTFGGGTKVEIKPAGGGGGSEPKSCDKTHTCPPCPAPELLGGPS
VFLFPPKPKDTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPGK
single chain (heavy) for h10G2 (h10U1G2)
SEQ. ID: 23
MEFGLSWVFLVALLRGVQCEVQLVESGGGLVQPGGSLRLSCAASGFTFSSYAMSWVRQTPGKGLEWVAVI
DSNGGSTYYPDTVKDRFTISRDNSKNTLYLQMNSLRAEDTAVYYCSSYTNLGAYWGQGTLVTVSAGGGGSG
GGGSGGGGSDIQMTQSPSSLSASVGDRVTITCRASQDISGYLNWLQQKPGGAIKRLIYTTSTLDSGVPKRFSG
SGSGTDFTLTISSLQSEDFATYYCLQYASSPFTFGGGTKVEIKPAGGGGGSERKCCVECPPCPAPPVAGPSVFLF
PPKPKDTLMISRTPEVTCVVVDVSHEDPEVQFNWYVDGVEVHNAKTKPREEQFNSTFRVVSVLTVVHQDW
LNGKEYKCKVSNKGLPAPIEKTISKTKGQPREPQVYTLPPSREEMTKNQVSLTCLVKGFYPSDISVEWESNGQ
PENNYKTTPPMLDSDGSFFLYSKLTVDKSRWQQGNVFSCSVMHEALHNHYTQKSLSLSPGK*
single chain (heavy) for h10G4 (h10U1G4)
SEQ. ID: 24
MEFGLSWVFLVALLRGVQCEVQLVESGGGLVQPGGSLRLSCAASGFTFSSYAMSWVRQTPGKGLEWVAVI
DSNGGSTYYPDTVKDRFTISRDNSKNTLYLQMNSLRAEDTAVYYCSSYTNLGAYWGQGTLVTVSAGGGGSG
GGGSGGGGSDIQMTQSPSSLSASVGDRVTITCRASQDISGYLNWLQQKPGGAIKRLIYTTSTLDSGVPKRFSG
SGSGTDFTLTISSLQSEDFATYYCLQYASSPFTFGGGTKVEIKPAGGGGGSEPKSCDKTHTCPPCPAPELLGGPS
VFLFPPKPKDTLMISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLGK
h5F6 scFv
SEQ. ID: 25
QVQLVQSGAEVKKPGASVKVSCKVSAYAFSSSWMNWVRQAPGKGLEWMGRIYPRDGDTNYNGKFKGRV
TMTADTSTDTAYMELSSLRSEDTAVYYCAREGDGYYWYFDVWGQGTTVTVSSGGGGSGGGGSGGGGSEI
VLTQSPATLSLSPGERATLSCRASQSIRNYLHWYQQKPGEAPRLLIYYASQSISGIPARFSGSGSGTDFTLTISSL
ETEDFAMYYCQHSNSWPLTFGQGTKLEIK
h10C12 scFv
SEQ. ID: 26
EVQLVESGGGLVQPGGSLRLSCAASGFTFSSYAMSWVRQTPGKGLEWVAVIDSNGGSTYYPDTVKDRFTISR
DNSKNTLYLQMNSLRAEDTAVYYCSSYTNLGAYWGQGTLVTVSAGGGGSGGGGSGGGGSDIQMTQSPSSL
SASVGDRVTITCRASQDISGYLNWLQQKPGGAIKRLIYTTSTLDSGVPKRFSGSGSGTDFTLTISSLQSEDFATY
YCLQYASSPFTFGGGTKVEIK
h9B5 scFv
SEQ. ID: 27
EVQLLESGGGLVQPGGSLRLSCAASGFTFSSYAMSWVRQAPGKGLEWVSVIDSNGGSTYYPDTVKDRFTISR
DNSKNTLYLQMNSLRAEDTAVYYCAKYTNLGAYWGQGTLVTVSSGGGGSGGGGSGGGGSDIQMTQSPSS
LSASVGDRVTITCRASQDISGYLNWYQQKPGKAPKLLIYTTSTLDSGVPSRFSGSGSGTDFTLTISSLQPEDFAT
YYCLQYASSPFTFGGGTKVEIK
h9C6 scFv
SEQ. ID: 28
QVQLVQSGAEVKKPGASVKVSCKVSGYTFTHYWMHWVRQRPGKGLEWMGEIDPFDSYTYYNQKFKGRVT
MTVDTSSDTAYMELSSLRSEDTAVYYCARPLPGTGWYFDVWGQGTTVTVSSGGGGSGGGGSGGGGSEIVL
TQSPTTLSLSPGERATLSCSASSSISSTYLHWYQQKPGFPPRLLIYGTSNLASGIPACFSGSGSGTDFTLTISSLEA
EDFAVYYCQQGSSLPFTFGQGTKLEIK
h8B5 scFv
SEQ. ID: 29
EVQLVESGGGLVQPGGSLRLSCAASGFTFSTYTMSWVRQTPAKGLVWVSTINSDGYNIYYSDSMKGRFTISR
DNAKYTLYLQMNSLRAEDTAMYYCARCSYYSYDYFDYWGQGTLVTVSSGGGGSGGGGSGGGGSDIQMTQ
SPSSLSASVGDRVTITCRASENIDNYLAWYQQKQGKVPKLLIYAATNLADGMPSRFSGSGSGTDFTLTISSLQP
EDVATYYCQHYYSNQLTFGQGTKLEIK
U1 scFv
SEQ. ID: 30
EVQLVESGGGLVQPGGSLRLSCAASGYSFTGYTMNWVRQAPGKGLEWVALINPYKGVSTYNQKFKDRFTIS
VDKSKNTAYLQMNSLRAEDTAVYYCARSGYYGDSDWYFDVWGQGTLVTVSSGGGGSGGGGSGGGGSDI
QMTQSPSSLSASVGDRVTITCRASQDIRNYLNWYQQKPGKAPKLLIYYTSRLESGVPSRFSGSGSGTDYTLTIS
SLQPEDFATYYCQQGNTLPWTFGQGTKVEIK
h5F6 CAR 2^{nd} Gen
SEQ. ID: 31
QVQLVQSGAEVKKPGASVKVSCKVSAYAFSSSWMNWVRQAPGKGLEWMGRIYPRDGDTNYNGKFKGRV
TMTADTSTDTAYMELSSLRSEDTAVYYCAREGDGYYWYFDVWGQGTTVTVSSGGGGSGGGGSGGGGSEI
VLTQSPATLSLSPGERATLSCRASQSIRNYLHWYQQKPGEAPRLLIYYASQSISGIPARFSGSGSGTDFTLTISSL
ETEDFAMYYCQHSNSWPLTFGQGTKLEIKGAPTTTPAPRPPTPAPTIASQPLSLRPEACRPAAGGAVHTRGL
DFACDIYIWAPLAGTCGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPQRRKNPQEGLYNELQKDKMAEAYS
EIGMKGERRRGKGHDGLYQGLSTATKDTYDALHMQALPPR*
h8B5 CAR 1st Gen
SEQ. ID: 32
EVQLVESGGGLVQPGGSLRLSCAASGFTFSTYTMSWVRQTPAKGLVWVSTINSDGYNIYYSDSMKGRFTISR
DNAKYTLYLQMNSLRAEDTAMYYCARCSYYSYDYFDYWGQGTLVTVSSGGGGSGGGGSGGGGSDIQMTQ
SPSSLSASVGDRVTITCRASENIDNYLAWYQQKQGKVPKLLIYAATNLADGMPSRFSGSGSGTDFTLTISSLQP
EDVATYYCQHYYSNQLTFGQGTKLEIKGAPTTTPAPRPPTPAPTIASQPLSLRPEACRPAAGGAVHTRGLDFA
CDIYIWAPLAGTCGVLLLSLVITLYCRVKFSRSADAPAYQQGQNQLYNELNLGRREEYDVLDKRRGRDPEMG
GKPQRRKNPQEGLYNELQKDKMAEAYSEIGMKGERRRGKGHDGLYQGLSTATKDTYDALHMQALPPR*
h10C12 CAR co-stimulatory
SEQ. ID: 33
EVQLVESGGGLVQPGGSLRLSCAASGFTFSSYAMSWVRQTPGKGLEWVAVIDSNGGSTYYPDTVKDRFTISR
DNSKNTLYLQMNSLRAEDTAVYYCSSYTNLGAYWGQGTLVTVSAGGGGSGGGGSGGGGSDIQMTQSPSSL
SASVGDRVTITCRASQDISGYLNWLQQKPGGAIKRLIYTTSTLDSGVPKRFSGSGSGTDFTLTISSLQSEDFATY
YCLQYASSPFTFGGGTKVEIKGAPTTTPAPRPPTPAPTIASQPLSLRPEACRPAAGGAVHTRGLDFACDIYIWA
PLAGTCGVLLLSLVITLYCKRGRKKLLYIFKQPFMRPVQTTQEEDGCSCRFPEEEEGGCEL*
h3Fc with mouse G1 Fc (ARB201)
SEQ. ID: 34
MEFGLSWVFLVALLRGVQCQVQLVESGGGVVQPGRSLRLSCAASGFTFSDYYMYWVRQAPGKGLEWVASI
SFDGTYTYYTDRVKGRFTISRDNSKNTLYLQMNSLRAEDTAVYYCARDRPAWFPYWGQGTLVTVSAGGGGS
GGGGSGGGGSGDIVMTQTPLSLSVTPGQPASISCRSSQSIVHSNGNTYLEWYLQKPGQSPQLLIYKVSNRFS
GVPDRFSGSGSGTDFTLKISRVEAEDVGVYYCFQGSHVPLTFGAGTKLELKPAGGGGGSEVQLVESGGGLVQ
PGGSLRLSCAASGYSFTGYTMNWVRQAPGKGLEWVALINPYKGVSTYNQKFKDRFTISVDKSKNTAYLQMN
SLRAEDTAVYYCARSGYYGDSDWYFDVWGQGTLVTVSSGGGGSGGGGSGGGGSDIQMTQSPSSLSASVG
DRVTITCRASQDIRNYLNWYQQKPGKAPKLLIYYTSRLESGVPSRFSGSGSGTDYTLTISSLQPEDFATYYCQQ
GNTLPWTFGQGTKVEIKGAPGGGSGEPKSSDKTHTCPPCPAPELLGGPSVFIFPPKPKDVLTITLTPKVTCVVV
DISKDDPEVQFSWFVDDVEVHTAQTKPREEQINSTFRSVSELPIMHQDWLNGKEFKCRVNSAAFPAPIEKTIS
KTKGRPKAPQVYTIPPPKEQMAKDKVSLTCMITNFFPEDITVEWQWNGQPAENYKNTQPIMDTDGSYFVYS
KLNVQKSNWEAGNTFTCSVLHEGLHNHHTEKSLSHSPGK
h3Fc with human G1 Fc
SEQ. ID: 35
MEFGLSWVFLVALLRGVQCQVQLVESGGGVVQPGRSLRLSCAASGFTFSDYYMYWVRQAPGKGLEWVASI
SFDGTYTYYTDRVKGRFTISRDNSKNTLYLQMNSLRAEDTAVYYCARDRPAWFPYWGQGTLVTVSAGGGGS
GGGGSGGGGSGDIVMTQTPLSLSVTPGQPASISCRSSQSIVHSNGNTYLEWYLQKPGQSPQLLIYKVSNRFS
GVPDRFSGSGSGTDFTLKISRVEAEDVGVYYCFQGSHVPLTFGAGTKLELKPAGGGGGSEVQLVESGGGLVQ
PGGSLRLSCAASGYSFTGYTMNWVRQAPGKGLEWVALINPYKGVSTYNQKFKDRFTISVDKSKNTAYLQMN
SLRAEDTAVYYCARSGYYGDSDWYFDVWGQGTLVTVSSGGGGSGGGGSGGGGSDIQMTQSPSSLSASVG
DRVTITCRASQDIRNYLNWYQQKPGKAPKLLIYYTSRLESGVPSRFSGSGSGTDYTLTISSLQPEDFATYYCQQ
GNTLPWTFGQGTKVEIKGAPGGGSGEPKSCDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK

What is claimed is:

1. An antibody having a N-terminal and a C-terminal, comprising a heavy chain and a light chain,
wherein the heavy chain comprises in tandem from the N-terminal to the C-terminal, a variable component comprising a heavy chain scFv domain, a heavy chain linker, a CH1, a hinge, a CH2 and a CH3 domain,
wherein the light chain comprises in tandem from the N-terminal to the C-terminal, a variable component comprising a light chain scFv domain, a light chain linker and a CL domain,
wherein the heavy chain scFv has a specificity against a first target,
wherein the light chain scFv has a specificity against a second target, and
wherein the first target and the second target are selected independently from a group comprising CDH17, CD3, TROP2, GPC3, and HER2.

2. An antibody having a N-terminal and a C-terminal, comprising,
a heavy chain comprising a scFv at the C terminus, and
a light chain,
wherein the heavy chain and the light chain form a Fab domain having a specificity against a first target,
wherein the scFv has a specificity against a second target, and
wherein the first target and the second target are selected independently from a group comprising CDH17, CD3, TROP2, GPC3, and HER2.

3. An antibody having a N-terminal and a C-terminal, comprising in tandem from the N-terminal to the C-terminal, a first scFv domain, a second scFv domain, a hinge, a CH2 domain and a CH3 domain,
wherein the first scFv domain has a specificity against a first target,
wherein the second scFv domain has a specificity against a second target, and
wherein the first target and the second target are selected independently from a group comprising CDH17, CD3, TROP2, GPC3, and HER2.

4. (canceled)

5. The antibody of claim 2, wherein CDH17 comprises CDH17 ectodomains D1, D2, D3, D4, D5, D6 and D7.

6. The antibody of claim 2, comprising an amino acid sequence having a homology of at least 98% with SEQ ID NO 15-33.

7. The antibody of claim 2, wherein the scFv domain has a specificity against CD3 or CD17.

8. The antibody of claim 2, wherein the Fab domain has a specificity against CD3 or CD17.

9-11. (canceled)

12. The antibody of claim 2, wherein the antibody is a mouse antibody, a humanized antibody, or a human antibody.

13. The antibody of claim 2, wherein the antibody is a human antibody isolated from a phage library screen.

14. The antibody of claim 2, further comprising a conjugated cytotoxic moiety.

15. The antibody of claim 14, wherein the conjugated cytotoxic moiety comprises irinotecan, a uristatins, PBDs, maytansines, amantins, spliceosome inhibitors, or a combination thereof.

16. The antibody of claims 14, wherein the conjugated cytotoxic moiety comprises a chemotherapeutic agent.

17. The antibody of claim 16, having a specificity for a cell receptor from a cytotoxic T or NK cell, or an immune checkpoint inhibitor.

18. The antibody of claim 17, wherein the immune checkpoint inhibitor comprises PD-1, TIM-3, LAG-3, TIGIT, CTLA-4, PD-L1, BTLA, VISTA, or a combination thereof.

19. The antibody of 17, having specificity for an angiogenic factor.

20. The antibody of claim 19, wherein the angiogenic factor comprises VEGF.

21-25. (canceled)

26. A pharmaceutical composition, comprising the antibody of claim 2 and a pharmaceutically acceptable carrier.

27. The pharmaceutical composition of claim 26, further comprising a cytotoxic agent, wherein the cytotoxic agent comprises cisplatin, gemcitabine, irinotecan, or an anti-tumor antibody.

28. (canceled)

29. A method for treating a subject having cancer, comprising administering to the subject an effective amount of the antibody of claim 2.

30. The method of claim 29, wherein the cancer is liver cancer, gastric cancer, colon cancer, pancreatic cancer, lung cancer, esophageal cancer or a combination thereof.
